(19) United States
(12) Patent Application Publication
(10) Pub. No.: US 2023/0263816 A1
(43) Pub. Date: Aug. 24, 2023

(54) PHARMACEUTICAL COMPOSITIONS FOR DELIVERY OF REMDESIVIR BY INHALATION

(71) Applicants: BOARD OF REGENTS, THE UNIVERSITY OF TEXAS SYSTEM, Austin, TX (US); TFF PHARMACEUTICALS, INC., Fort Worth, TX (US)

(72) Inventors: Robert O. WILLIAMS, III, Austin, TX (US); Chaeho MOON, Austin, TX (US); Sawittree SAHAKIJPIJARN, Austin, TX (US); Dale CHRISTENSEN, Fort Worth, TX (US); Glenn MATTES, Fort Worth, TX (US); John J. KOLENG, Austin, TX (US)

(21) Appl. No.: 18/005,522

(22) PCT Filed: Jul. 16, 2021
(86) PCT No.: PCT/US2021/042010

§ 371 Date: Jan. 13, 2023

Related U.S. Application Data
(60) Provisional application No. 63/053,339, filed on Jul. 17, 2020.

(51) Int. Cl. A61K 31/706; A61K 9/00; A61K 47/18; A61K 47/26; A61K 47/40; A61K 45/06

(52) U.S. Cl. CPC A61K 31/706; A61K 9/0075; A61K 47/183; A61K 47/26; A61K 47/40; A61K 45/06

(57) ABSTRACT

The present disclosure provides pharmaceutical compositions of remdesivir that may be administered by inhalation. These compositions may allow the achievement of a therapeutically effective dose of remdesivir directly to the lungs. These compositions may be used to treat one or more diseases or disorders such as a viral infection like an infection of a coronavirus.

This invention claims the benefit of priority to U.S. Provisional Application No. 63/053,339, filed on Jul. 17, 2020, the entire contents of which are hereby incorporated by reference.

### BACKGROUND

### 1. Field

The present disclosure relates generally to the field of pharmaceuticals and pharmaceutical manufacture. More particularly, it concerns compositions and methods of preparing a pharmaceutical composition for inhalation comprising remdesivir.

### 2. Description of Related Art

The WHO has declared the Coronavirus Disease 2019 (COVID-19) outbreak a pandemic (WHO Director-General's opening remarks at the Mission briefing on COVID-19—12 Mar. 2020. www.who.int/dg/speeches/detail/who-director-general-s-opening-remarks-at-the-mission-briefing-on-covid-19—12-march-2020). This virus is related to other coronaviruses that have created pandemics called the Severe Acute Respiratory Syndrome (SARS-CoV) in 2002 and the Middle East Respiratory Syndrome (MERS-CoV) in 2012 (Wu et al., 2004; Peeri et al., 2020). Currently, this COVID-19 has killed more people than the others two mentioned pandemics together (Gurwitz, 2020). This COVID-19 has been named as SARS-CoV-2 because its share near 80% of the genome with the SARS-CoV (Yan et al., 2020). Moreover, it has been reported that both viruses interact with similar affinity with angiotensin-converting enzyme 2 (ACE2), a protein that works as an entry receptor (Ahmed et al., 2020; Walls et al., 2020).

Unfortunately, there are no specific drugs or vaccines for coronaviruses (Walls et al., 2020; Tortoric et al., 2019). The present strategy in drug discovery has been the test of drugs previously used in SARS and MERS (Wang et al., 2020) or other coronaviruses. Recently, remdesivir administered by injection has been shown in numerous clinical studies to reduce the time to recovery in hospitalized patients with COVID-19 (Beigel et al., 2020)

While remdesivir has been shown to reduce the time spent in the hospital, the current formulations require intravenous infusion of the remdesivir in a hospital or medical facility. This limited administration route prevents the use of remdesivir early within the infection cycle when it may be useful in preventing hospitalization of the patients. Furthermore, delivery via inhalation allows the administration of the drug directly to the most effective organs. Therefore, there remains a need for improved formulations of remdesivir for potential treatment of numerous indications.

### SUMMARY OF THE INVENTION

The present disclosure provides pharmaceutical compositions comprising remdesivir for administration via inhalation. Without wishing to be bound by any theory, these compositions may have one or more advantageous properties such as higher drug loading, maintenance of a therapeutically effective dose, or other properties such as ability to more effectively deliver the drug to the target organ. In some embodiments, the present disclosure provides pharmaceutical compositions comprising:

(A) an active pharmaceutical ingredient wherein active pharmaceutical ingredient is remdesivir or a pharmaceutically acceptable salt thereof;

wherein the pharmaceutical composition is formulated for administration via inhalation and the pharmaceutical composition is a dry powder.

In some embodiments, the pharmaceutical composition is a brittle matrix particle. In some embodiments, the pharmaceutical composition comprises one or more nanoparticles. In some embodiments, the pharmaceutical composition comprises at least 75% of the active pharmaceutical ingredient in an amorphous form. In some embodiments, at least 90% of the active pharmaceutical ingredient is in the amorphous form. In some embodiments, at least 95% of the active pharmaceutical ingredient is in the amorphous form. In some embodiments, at least 98% of the active pharmaceutical ingredient is in the amorphous form. In some embodiments, at least 99% of the active pharmaceutical ingredient is in the amorphous form. In some embodiments, the pharmaceutical composition comprises no crystalline active pharmaceutical ingredient.

In some embodiments, the pharmaceutical composition further comprises an excipient. In some embodiments, the excipient is in the crystalline form. In other embodiments, the excipient is in the amorphous form.

In some embodiments, the excipient is an amino acid. In some embodiments, the amino acid is a hydrophobic amino acid such as leucine. In other embodiments, the excipient is a sugar or sugar derivative. In some embodiments, the sugar is a sugar alcohol such as mannitol. In other embodiments, the sugar is a monosaccharide or disaccharide. In some embodiments, the sugar is a disaccharide such as lactose. In other embodiments, the excipient is a cyclodextrin. In some embodiments, the cyclodextrin is a β-cyclodextrin. In some embodiments, the cyclodextrin is modified with one or more sulfonate groups. In some embodiments, the sulfonate groups are sulfonate salts. In some embodiments, the sulfonate salts are alkali metal salts such as sodium salts. In some embodiments, the cyclodextrin and the sulfonate groups are connected by an ether spacer. In some embodiments, the ether spacer is a butyl ether spacer. In some embodiments, the excipient is Captisol® or Dexolve™.

In some embodiments, the pharmaceutical composition comprises from about 1% w/w to about 99% w/w of the active pharmaceutical ingredient. In some embodiments, the pharmaceutical composition comprises from about 5% w/w to about 95% w/w of the active pharmaceutical ingredient. In some embodiments, the pharmaceutical composition comprises from about 10% w/w to about 90% w/w of the active pharmaceutical ingredient. In some embodiments, the pharmaceutical composition comprises from about 5% w/w to about 45% w/w of the active pharmaceutical ingredient. In other embodiments, the pharmaceutical composition comprises from about 45% w/w to about 90% w/w of the active pharmaceutical ingredient.

In some embodiments, the pharmaceutical composition comprises from about 5% w/w to about 95% w/w of the excipient. In some embodiments, the pharmaceutical composition comprises from about 5% w/w to about 45% w/w of the excipient. In other embodiments, the pharmaceutical composition comprises from about 45% w/w to about 90% w/w of the excipient.

In some embodiments, the pharmaceutical composition is substantially free of any other compound other than the active pharmaceutical ingredient. In some embodiments, the pharmaceutical composition is essentially free of any other compound other than the active pharmaceutical ingredient. In some embodiments, the pharmaceutical composition is entirely free of any other compound other than the active pharmaceutical ingredient.

In some embodiments, the pharmaceutical composition has a median mass aerodynamic diameter from about 0.1 μm to about 10 μm. In some embodiments, the median mass aerodynamic diameter is from about 0.25 μm to about 5 μm. In some embodiments, the median mass aerodynamic diameter is from about 0.5 μm to about 3.5 μm. In some embodiments, the median mass aerodynamic diameter is from about 0.75 μm to about 3 μm.

In some embodiments, the pharmaceutical composition comprises a geometric standard deviation is from about 0.1 to about 5. In some embodiments, the geometric standard deviation is from about 0.5 to about 4.5. In some embodiments, the geometric standard deviation is from about 1 to about 4. In some embodiments, the geometric standard deviation is from about 2 to about 3.75. In some embodiments, the geometric standard deviation is from about 2 to about 3.

In some embodiments, the pharmaceutical composition has a delivered fine particle fraction of greater than 50%. In some embodiments, the delivered fine particle fraction is greater than 60%. In some embodiments, the delivered fine particle fraction is greater than 70%. In some embodiments, the delivered fine particle fraction is greater than 80%. In some embodiments, the pharmaceutical composition has a recovered fine particle fraction of greater than 50%. In some embodiments, the recovered fine particle fraction is greater than 60%. In some embodiments, the recovered fine particle fraction is greater than 70%. In some embodiments, the recovered fine particle fraction is greater than 80%. In some embodiments, the pharmaceutical composition has an emitted dose of greater than 75%. In some embodiments, the emitted dose is greater than 80%. In some embodiments, the emitted dose is greater than 85%. In some embodiments, the emitted dose is greater than 90%.

In some embodiments, the pharmaceutical composition is substantially free of any lubricants, antistatic agents, anti-adherents, glidants, peptides, surfactants, lipids, and phospholipids. In some embodiments, the pharmaceutical composition is essentially free of any lubricants, antistatic agents, anti-adherents, glidants, peptides, surfactants, lipids, and phospholipids. In some embodiments, the pharmaceutical composition is entirely free of any lubricants, antistatic agents, anti-adherents, glidants, peptides, surfactants, lipids, and phospholipids. In some embodiments, the pharmaceutically composition is substantially free of any added excipients. In some embodiments, the pharmaceutical composition is essentially free of any added excipients. In some embodiments, the pharmaceutical composition is entirely free of any added excipients. In some embodiments, the pharmaceutical composition is substantially free of any excipients. In some embodiments, the pharmaceutical composition is essentially free of any excipients. In some embodiments, the pharmaceutical composition is entirely free of any excipients.

In some embodiments, the pharmaceutical composition is loaded into an inhaler such as a dry powder inhaler, a metered dose inhaler, a single dose inhaler, a multi-dose inhaler, or a pressurized metered dose inhaler. In some embodiments, the pharmaceutical composition is formulated as unit dose. In some embodiments, the unit dose is formulated for dry powder inhalation as a capsule, cartridge, or blister. In some embodiments, the capsule, cartridge, or blister is designed for use with a dry powder inhaler.

In some embodiments, the pharmaceutical composition is present in a container which blocks UV light. In some embodiments, the pharmaceutical composition is present in a container which blocks moisture uptake. In some embodiments, the pharmaceutical composition is present in a container which blocks oxygen ingress. In some embodiments, the pharmaceutical composition is present in a container which desiccates the composition.

In some embodiments, the composition further comprises a second active pharmaceutical ingredient. In some embodiments, the second active pharmaceutical ingredient is anti-inflammatory such as beclomethasone, budesonide, dexamethasone, ciclesonide, fluticasone, mometasone, prednisone, methylprednisone, a statin, or clofazimine. In other embodiments, the second active pharmaceutical ingredient is anti-microbial such as niclosamide, ivermectin, chloroquine, hydroxychloroquine, lopinavir, or favipiravir. In other embodiments, the second active pharmaceutical ingredient is an antibody. In other embodiments, the second active pharmaceutical ingredient is an immunomodulatory therapy such as tocilizumab, sarilumab, anakinra, or ruxolitinib. In other embodiments, the second active pharmaceutical ingredient is an anticoagulant such as heparin. In other embodiments, the second active pharmaceutical ingredient is an antifibrotic such as a tyrosine kinase inhibitor.

In still yet another aspect, the present disclosure provides inhalers comprising a pharmaceutical composition described herein.

In yet another aspect, the present disclosure provides methods of preparing a dry powder pharmaceutical composition described herein comprising:

(A) dissolving an active pharmaceutical ingredient, wherein the active agent is remdesivir or a pharmaceutically acceptable salt thereof, in a solvent to obtain a pharmaceutical mixture;
(B) applying the pharmaceutical mixture to a surface at a surface temperature below 0° C. to obtain a frozen pharmaceutical mixture; and
(C) collecting the frozen pharmaceutical mixture and drying the frozen pharmaceutical mixture to obtain a dry powder pharmaceutical composition.

In some embodiments, the solvent is an organic solvent such as acetonitrile, tert-butanol, or 1,4-dioxane. In some embodiments, the methods further comprise admixing the active pharmaceutical ingredient with an excipient. In some embodiments, the pharmaceutical mixture further comprises a second solvent. In some embodiments, the excipient is dissolved in the second solvent and then added to the pharmaceutical mixture. In some embodiments, the second solvent is water. In some embodiments, the first solvent is mixed with the second solvent to obtain a homogenous pharmaceutical mixture. In some embodiments, the pharmaceutical mixture is admixed until the pharmaceutical mixture is clear.

In some embodiments, the pharmaceutical mixture comprises a solid content from about 0.05% w/v to about 5% w/v of the active pharmaceutical ingredient and the excipient. In some embodiments, the solid content is from about 0.1% w/v to about 2.5% w/v of the active pharmaceutical ingredient and the excipient. In some embodiments, the solid content is from about 0.15% w/v to about 1.5% w/v of the active pharmaceutical ingredient and the excipient. In some embodiments, the solid content is from about 0.2% w/v to about 0.6% w/v of the active pharmaceutical ingredient and the excipient. In other embodiments, the solid content is from about 0.5% w/v to about 1.25% w/v of the active pharmaceutical ingredient and the excipient.

In some embodiments, the pharmaceutical mixture is applied at a feed rate from about 0.5 mL/min to about 5 mL/min. In some embodiments, the feed rate is from about 1 mL/min to about 3 mL/min In some embodiments, the feed rate is about 2 mL/min. In some embodiments, the pharmaceutical mixture is applied with a nozzle such as a needle like a 19 gauge needle. In some embodiments, the pharmaceutical mixture is applied from a height from about 2 cm to about 50 cm. In some embodiments, the height is from about 5 cm to about 20 cm such as about 10 cm.

In some embodiments, the surface temperature is from about 0° C. to −190° C. In some embodiments, the surface temperature is from about −25° C. to about −125° C. such as about −100° C. In some embodiments, the surface is a rotating surface. In some embodiments, the surface is rotating at a speed from about 5 rpm to about 500 rpm. In some embodiments, the surface is rotating at a speed from about 100 rpm to about 400 rpm such as at a speed of about 200 rpm.

In some embodiments, the frozen pharmaceutical composition is dried by lyophilization. In some embodiments, the frozen pharmaceutical composition is dried at a first reduced pressure. In some embodiments, the first reduced pressure is from about 10 mTorr to 500 mTorr. In some embodiments, the first reduced pressure is from about 50 mTorr to about 250 mTorr such as about 100 mTorr. In some embodiments, the frozen pharmaceutical composition is dried at a first reduced temperature. In some embodiments, the first reduced temperature is from about 0° C. to −100° C. In some embodiments, the first reduced temperature is from about −20° C. to about −60° C. such as about −40° C. In some embodiments, the frozen pharmaceutical composition is dried for a primary drying time period from about 3 hours to about 36 hours. In some embodiments, the primary drying time period is from about 6 hours to about 24 hours such as about 20 hours.

In some embodiments, the frozen pharmaceutical composition is dried a secondary drying time period. In some embodiments, the frozen pharmaceutical composition is dried a secondary drying time at a second reduced pressure. In some embodiments, the secondary drying time is at a reduced pressure is from about 10 mTorr to 500 mTorr. In some embodiments, the secondary drying time is at a reduced pressure is from about 50 mTorr to about 250 mTorr such as about 100 mTorr. In some embodiments, the frozen pharmaceutical composition is dried a secondary drying time at a second reduced temperature. In some embodiments, the second reduced temperature is from about 0° C. to 30° C. In some embodiments, the second reduced temperature is from about 10° C. to about 30° C. such as about 25° C. In some embodiments, the frozen pharmaceutical composition is dried for a second time for a second time period from about 3 hours to about 36 hours. In some embodiments, the second time period is from about 6 hours to about 24 hours such as about 20 hours.

In still yet another aspect, the present disclosure provides pharmaceutical compositions prepared by the method described herein.

In yet another aspect, the present disclosure provides methods of treating a disease or disorder in a patient comprising administering a pharmaceutical composition described herein to the patient in a therapeutically effective amount. In some embodiments, the disease or disorder is a microbial infection such as a viral infection. In some embodiments, the viral infection is an infection of a coronavirus such as MERS-Cov, SARS-Cov1, or SARS-Cov2 (COVID-19). In some embodiments, the active pharmaceutical ingredient is inhaled into the lungs. In some embodiments, the active pharmaceutical ingredient is inhaled into the alveolar sacs within the lungs. In some embodiments, the patient is exhibiting one or more symptoms of a viral infection.

In some embodiments, the patient is not yet hospitalized. In other embodiments, the patient has been hospitalized. In some embodiments, the patient is not yet receiving supplemental oxygen. In some embodiments, the patient is not yet receiving mechanical ventilation. In some embodiments, the patient has not yet been diagnosed with a viral infection. In some embodiments, the patient has been exposed to a person exhibiting one or more symptoms of a viral infection. In other embodiments, the patient has been exposed to a person who has been diagnosed with a viral infection. In some embodiments, the patient is administered the pharmaceutical composition prophylactically. In some embodiments, the patient has been diagnosed with a viral infection. In some embodiments, the viral infection is an infection of a coronavirus such as SARS-Cov2.

In some embodiments, the methods comprise administering a dose from about 1 mg to about 250 mg. In some embodiments, the dose is from about 5 mg to about 100 mg. In some embodiments, the dose is from about 7.5 mg to about 75 mg. In some embodiments, the dose is from about 10 mg to about 30 mg.

In still yet another aspect, the present disclosure provides methods of reducing lung inflammation in a patient comprising administering a pharmaceutical composition described herein to the patient in a therapeutically effective amount. In some embodiments, the lung inflammation is associated with a viral infection. In some embodiments, the pharmaceutical composition is administered once. In other embodiments, the pharmaceutical composition is administered more than once. In some embodiments, the pharmaceutical composition is administered at a first dose and then administered again at a different second dose. In some embodiments, the first dose is greater than the second dose.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A & 1B show the brittle matrix particle morphology of TFF remdesivir compositions; (FIG. 1A) formulations prepared in acetonitrile/water (50/50) co-solvent mixture; (FIG. 1B) formulations prepared in 1,4-dioxane/water (50/50) co-solvent mixture.

FIGS. 2A & 2B show the X-ray powder diffraction (XRPD) diffractograms of TFF remdesivir compositions; (FIG. 2A) formulations prepared in acetonitrile/water (50/50) solvent mixture; (FIG. 2B) formulations prepared in 1,4-dioxane/water (50/50) solvent mixture.

FIGS. 3A & 3B show the modulated differential scanning calorimetry (mDSC) thermograms; (FIG. 3A) remdesivir unprocessed powder; (FIG. 3B) TFF remdesivir powder formulations.

FIGS. 4A & 4B show the Aerodynamic diameter distribution of TFF remdesivir formulations when emitted from a Plastiape® RS00 high-resistance DPI at a flow rate of 60 L/min (n=3); (FIG. 4A) formulations prepared in acetonitrile/water (50/50) co-solvent mixture; (FIG. 4B) formulations prepared in 1,4-dioxane/water (50/50) co-solvent mixture.

FIGS. 5A-5C show the ¹H-NMR spectra; (FIG. 5A) TFF remdesivir powder formulations (F7, F8, F9, F10, F14) at initial condition, TFF remdesivir powder formulations (F10, F12, F13) at 25° C./60% RH at 1 month, and remdesivir unprocessed powder (remdesivir); (FIG. 5B) expansion of TFF remdesivir powder formulations (F7, F8, F9, F10, F14) and remdesivir unprocessed powder (remdesivir); (FIG. 5C) expansion of TFF remdesivir powder (F9) and leucine unprocessed powder (leucine).

FIG. 6 shows the XRD diffractograms of F10, F12, and F13 after storage at 25° C./60% RH.

FIG. 7 shows the aerosol performance of TFF remdesivir powder formulations after one month of storage at 25° C./60% RH when emitted from a Plastiape® RS00 high-resistance DPI at a flow rate of 60 L/min (n=3).

FIG. 8 shows the dissolution profiles of TFF remdesivir dry powder compositions in simulated lung fluid.

FIGS. 9A & 9B shows the plasma concentration-time profiles of F10 (remdesivir-Captisol®; 80/20 w/w) and F13 (remdesivir-leucine; 80/20 w/w) after a single inhalation administration in rats; (FIG. 9A) remdesivir; (FIG. 9B) GS-441524.

FIGS. 10A & 10B show the lung concentration-time profiles of REM-CAP (remdesivir-Captisol®; 80/20 w/w) and REM-LEU (remdesivir-leucine; 80/20 w/w) after a single inhalation administration in hamsters; (FIG. 10A) remdesivir; (FIG. 10B) GS-441524.

FIGS. 11A & 11B show the plasma concentration-time profiles of REM-CAP (remdesivir-Captisol®; 80/20 w/w) and REM-LEU (remdesivir-leucine; 80/20 w/w) after a single inhalation administration in hamsters; (FIG. 11A) remdesivir; (FIG. 11B) GS-441524. Dash line and dot line represent EC50 of remdesivir and GS-441524 in human epithelial cells (HAE) (Agostini et al., 2018), and continuous human lung epithelial cell line (Calu-3) (Gilead Sciences, Inc., 2020), respectively.

### DESCRIPTION OF ILLUSTRATIVE EMBODIMENTS

In some aspects of the present disclosure, the pharmaceutical compositions provided herein may comprise remdesivir in formulations for administration via inhalation. In some aspects, the present compositions may be used to deliver a therapeutically effective dose directly to the lungs. By delivering these compositions directly to the lungs, it may reduce the amount of active pharmaceutical ingredient needed or may allow administration outside of a doctor's office or hospital. The present disclosure also provides methods of preparing these compositions or uses of these compositions to treat a disease or disorder such as a microbial infection.

Also provided herein are methods of preparing and using these compositions. Details of these compositions are provided in more detail below.

### I. PHARMACEUTICAL COMPOSITIONS

In some aspects, the present disclosure provides pharmaceutical compositions containing an active pharmaceutical ingredient, such as remdesivir, and may optionally contain an excipient formulated for administration via inhalation.

### A. Remdesivir

The pharmaceutical compositions described herein comprise remdesivir as an active agent. In some embodiments, the pharmaceutical composition is free from any compound other than remdesivir. In other embodiments, the pharmaceutical compositions described herein contain remdesivir in an amount between about 5% to about 95% w/w, between about 20% to about 80% w/w, or between about 40% to about 60% w/w of the total composition. In some embodiments, the amount of the remdesivir is from about 5%, 10%, 20%, 30%, 35%, 40%, 45%, 50%, 55%, 60%, 65%, 70%, 75%, 80%, to about 90% w/w or any range derivable therein.

### 1. Inhalation

In some embodiments, the present disclosure relates to respirable particles that must be in the aerodynamic size range of around 0.25 to 5 microns or 0.5 to 3.5 microns in aerodynamic diameter. Typical approaches to obtain particles of this size range are by air jet milling, spray drying, thin-film freezing, and other methods known in the art. Regardless of the method used to achieve drug particles in the appropriate size range, micronization results in highly cohesive particles that are generally resistant to aerosol dispersion. This is typically overcome through formulation as interactive mixtures with larger lactose carrier particles (e.g., 45-75 μm) or other dispersion enhancing excipients. Drug particles are admixed by blending with appropriate carrier(s) particles using conventional mixing, or the drug and carrier can be prepared simultaneously from a solution or suspension formulation such as by thin film freezing processes, spray drying, milling, or other formulation techniques.

In some embodiments, the present disclosure provides methods for the administration of the inhalable remdesivir composition provided herein using a device. Administration may be, but is not limited, to inhalation of remdesivir using an inhaler. In some embodiments, an inhaler is a simple passive dry powder inhaler (DPI), such as a Plastiape RS01 monodose DPI. In a simple dry powder inhaler, dry powder is stored in a capsule or reservoir and is delivered to the lungs by inhalation without the use of propellants.

In some embodiments, an inhaler is a single-dose DPI, such as a DoseOne™ Spinhaler, Rotohaler®, Aerolizer®, or Handihaler. In some embodiments, an inhaler is a multidose DPI, such as a Plastiape RS02, Turbuhaler®, Twisthaler™, Diskhaler®, Diskus®, or Ellipta™. In some embodiments, the inhaler is Twincer®, Orbital®, TwinCaps®, Powdair, Cipla Rotahaler, DP Haler, Revolizer, Multi-haler, Twister, Starhaler, or Flexhaler®. In some embodiments, an inhaler is a plurimonodose DPI for the concurrent delivery of single doses of multiple medications, such as a Plastiape RS04 plurimonodose DPI. Dry powder inhalers have medication stored in an internal reservoir, and medication is delivered by inhalation with or without the use of propellants. Dry powder inhalers may require an inspiratory flow rate greater than 30 L/min for effective delivery, such as between about 30-120 L/min.

In some embodiments, the inhalable remdesivir is delivered as a propellant formulation, such as HFA propellants.

In some embodiments, the inhaler may be a metered dose inhaler. Metered dose inhalers deliver a defined amount of medication to the lungs in a short burst of aerosolized medicine aided by the use of propellants. Metered dose inhalers comprise three major parts: a canister, a metering valve, and an actuator. The medication formulation, including propellants and any required excipients, are stored in the canister. The metering valve allows a defined quantity of the medication formulation to be dispensed. The actuator of the metered dose inhaler, or mouthpiece, contains the mating discharge nozzle and typically includes a dust cap to prevent contamination.

In some embodiments, an inhaler is a nebulizer. A nebulizer is used to deliver medication in the form of an aerosolized mist inhaled into the lungs. The medication formulation is aerosolized by compressed gas, or by ultrasonic waves. A jet nebulizer is connected to a compressor. The compressor emits compressed gas through a liquid medication formulation at a high velocity, causing the medication formulation to aerosolize. Aerosolized medication is then inhaled by the patient. An ultrasonic wave nebulizer generates a high frequency ultrasonic wave, causing the vibration of an internal element in contact with a liquid reservoir of the medication formulation, which causes the medication formulation to aerosolize. Aerosolized medication is then inhaled by the patient. A nebulizer may utilize a flow rate of between about 3-12 L/min, such as about 6 L/min. In some embodiments, the nebulizer is a dry powder nebulizer.

In some embodiments, the composition may be administered on a routine schedule. As used herein, a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In some embodiments, remdesivir is administered once per day. In preferred embodiments, remdesivir is administered less than once per day, such as every other day, every third day, or once per week. In some embodiments, a therapeutic dose of remdesivir is between 1-250 mg, such as 2.5-150, 5-100, 5-50, or 10-30 mg. The therapeutic dose may be made up of one or more unit doses administered at one or more times per dosing period.

In some embodiments, remdesivir may be provided in a unit dosage form, such as in a capsule, blister or a cartridge, wherein the unit dose comprises at least 1 mg of remdesivir, such as at least 1 mg or 10 mg of remdesivir per dose. In particular aspects, the unit dosage form does not comprise the administration or addition of any excipient and is merely used to hold the powder for inhalation (i.e., the capsule, blister, or cartridge is not administered). In some embodiments, remdesivir may be administered in a high emitted therapeutic dose, such as at least 1 mg, preferably at least 5 mg, even more preferably 7.5 mg. In some embodiments, administration of remdesivir results in a high fine particle therapeutic dose into the deep lung such as greater than 1 mg. Preferably, the fine particle therapeutic dose into the deep lung is at least 1 mg, even more preferably at least 5 mg.

In some embodiments, the compositions have aerodynamic parameters including a mass median aerodynamic diameter from about 0.1 μm to about 10 μm, from about 0.25 μm to about 5 μm, from about 0.5 μm to about 3.5 μm, or from about 0.75 μm to about 3 μm. The mass median aerodynamic diameter is from about 0.1 μm, 0.25 μm, 0.5 μm, 0.75 μm, 1 μm, 1.25 μm, 1.5 μm, 1.75 μm, 2 μm, 2.25 μm, 2.5 μm, 2.75 μm, 3 μm, 3.25 μm, 3.5 μm, 3.75 μm, 4 μm, 5 μm, to about 10 μm, or any range derivable therein. Similarly, the composition may have a geometric standard deviation from about 0.1 to about 10, from about 0.1 to about 0.5, from about 0.5 to about 4.5, from about 1 to about 4, from about 2 to about 3.75, or from about 2 to about 3. The geometric standard deviation is from about 0.1 μm, 0.5 μm, 1 μm, 1.5 μm, 1.75 μm, 2 μm, 2.1 μm, 2.2 μm, 2.25 μm, 2.3 μm, 2.4 μm, 2.5 μm, 2.6 μm, 2.7 μm, 2.75 μm, 2.8 μm, 2.9 μm, 3 μm, 3.25 μm, 3.5 μm, 3.75 μm, 4 μm, 5 μm, to about 10 μm, or any range derivable therein. These compositions may result in a delivered or recovered fine particle fraction of greater than 50%, greater than 60%, greater than 70%, or greater than 80%. Similarly, the composition may result in an emitted dose from an inhaler of greater than 60%, of greater than 70%, of greater than 75%, of greater than 80%, of greater than 85%, or of greater than 90%,

In some embodiments, changes in pressure drop across the device result in a change in emitted dose. In some embodiments, changes in pressure drop across the device of 3 kPa, such as from 4 kPa to 1 kPa, result in a reduction of emitted dose of less than 35%, such as 34%, 33%, 32%, 31%, 30%, 29%, 28%, 27%, 26%, 25%, 24%, 23%, 22%, 21%, 20%, 19%, 18%, 17%, 16%, 15% or less. In some embodiments, changes in inhalation pressure drop across the device result in a change in fine particle dose. In some embodiments, changes in inhalation pressure drop across the device of 3 kPa, such as from 4kPa to 1 kPa result in a reduction of fine particle dose of less than 35%, such as 34%, 33%, 32%, 31%, 30%, 29%, 28%, 27%, 26%, 25%, 24%, 23%, 22%, 21%, 20%, 19%, 18%, 17%, 16%, 15% or less.

### B. Excipients

In some aspects, the present disclosure comprises one or more excipients formulated into pharmaceutical compositions. An “excipient” refers to pharmaceutically acceptable carriers that are relatively inert substances used to facilitate administration or delivery of an API into a subject or used to facilitate processing of an API into drug formulations that can be used pharmaceutically for delivery to the site of action in a subject. Non-limiting examples of excipients include diluents, carriers, stabilizing agents, surfactants, surface modifiers, solubility enhancers, buffers, encapsulating agents, antioxidants, preservatives, nonionic wetting or clarifying agents, viscosity increasing agents, and absorption-enhancing agents. In some embodiments, if the composition further comprises an excipient, then the excipient and the active pharmaceutical ingredient are contained within the same drug particle.

In some aspects, the amount of the excipient in the pharmaceutical composition is from about 5% to about 95% w/w, from about 20% to about 80% w/w, from about 30% to about 70% w/w, or from about 40% to about 60% w/w. The amount of the excipient in the pharmaceutical composition comprises from about 5%, 10%, 15%, 20%, 25%, 30%, 35%, 40%, 45%, 50%, 55%, 60%, 65%, 70%, 75%, 80%, 85%, 90%, to about 95% w/w, or any range derivable therein, of the total pharmaceutical composition. In one embodiment, the amount of the excipient in the pharmaceutical composition is at 40% to 60% w/w of the total weight of the pharmaceutical composition.

In some aspects, the present disclosure may further comprise one or more excipient such as a saccharide, a sugar derivative, an amino acid, or a cyclodextrin. Some composition may further comprise a mixture of two or more excipients.

### 1. Amino Acids, Sugars, Saccharides, and Hydrophobic Carriers

In some aspects, the present disclosure comprises one or more excipients formulated into pharmaceutical compositions. In some embodiments, the excipients used herein are water soluble excipients. These water-soluble excipients include carbohydrates or saccharides, such as disaccharides such as sucrose, trehalose, or lactose, a trisaccharide such as fructose, glucose, galactose comprising raffinose, polysaccharides such as starches or cellulose, or a sugar alcohol such as xylitol, sorbitol, or mannitol. In some embodiments, these excipients are solid at room temperature. Some non-limiting examples of sugar alcohols include erythritol, threitol, arabitol, xylitol, ribitol, mannitol, sorbitol, galactitol, fucitol, iditol, inositol, volemitol, isomalt, maltitol, lactitol, maltotritol, maltotetraitol, or a polyglycitol. In other aspects, larger molecules like amino acids, peptides and proteins are incorporated to facilitate inhalation delivery, including leucine, trileucine, histidine and others. In some embodiments, the amino acid may be an amino acid without any polar functional groups such as carboxylic acid, amine, amide, or guanidine functional group on the side chain of the amino acid. Some non-limiting examples of hydrophobic amino acids include glycine, proline, leucine, isoleucine, valine, alanine, phenylalanine, tryptophan, or tyrosine. The amino acids that may be used herein include any one of the canonical amino acids such as leucine, histidine, cysteine, lysine, or tyrosine. In other embodiments, the pharmaceutical composition may further comprise a cyclodextrin or a cyclodextrin derivative. The cyclodextrin may be an α-cyclodextrin, a β-cyclodextrin, or a γ-cyclodextrin, wherein the cyclodextrin is comprises of 5 or more α-D-glucopyranoside units that are joined by 1-4 linkages. These cyclodextrin compounds may be further derivatized with one or more groups that modify the compounds such that they are more water soluble or modify their chemical properties. These group may include a sulfonate group, an acetyl group, a methyl group, or a polymer group such as a polyethylene glycol or a polypropylene glycol.

When an excipient is present in the composition, the excipient is present in the composition at a level between 1% to 90% w/w, between 10% to 80% w/w, between 20% to 70% w/w, between 30% to 70% w/w, between 40% to 60% w/w. In some embodiments, the amount of the excipient is from about 1%, 2%, 5%, 10%, 15%, 50%, 20%, 25%, 30%, 35%, 37.5%, 40%, 41%, 42%, 43%, 44%, 45%, 46%, 47%, 48%, 49%, 50%, 51%, 52%, 53%, 54%, 55%, 56%, 57%, 58%, 59%, 60%, 65%, 70%, 75%, 80%, 85%, 90%, to about 95% w/w or any range derivable therein.

### II. MANUFACTURING METHODS

### A. Thin Film Freezing

Thus, in one aspect, the present disclosure provides pharmaceutical compositions which may be prepared using a thin-film freezing process. Methods of preparing pharmaceutical compositions using thin film freezing are described in U.S. Patent Application No. 2010/0221343, Watts, et al., 2013, Engstrom et al. 2008, Wang et al. 2014, Thakkar at el. 2017, O'Donnell et al. 2013, Lang et al. 2014a, Lang et al. 2014b, Carvalho et al. 2014, Beinborn et al. 2012a, Beinborn et al. 2012b, Zhang et al. 2012, Overhoff et al. 2009, Overhoff et al. 2008, Overhoff et al. 2007a, Overhoff et al. 2007b, Watts et al. 2010, Yang et al. 2010, DiNunzio et al. 2008, Purvis et al. 2007, Liu et al. 2015, Sinswat et al. 2008, and U.S. Pat. No. 8,968,786, all of which are incorporated herein by reference. In some embodiments, these methods involve dissolving the components of the pharmaceutical composition into a solvent to form a pharmaceutical mixture. The solvents may be either water, an organic solvent, or a mixture thereof. Some non-limiting examples of organic solvents which may be used include volatile organic solvent such as 1,4-dioxane, acetonitrile, acetone, methanol, ethanol, isopropanol, dichloromethane, chloroform, tetrahydrofuran, tert-butyl alcohol, dimethyl sulfoxide, N,N-dimethyl formamide, diethyl ether, ethyl acetate, isopropyl acetate, butyl acetate, propyl acetate, toluene, hexanes, heptane, pentane, or combinations thereof. In some embodiments, the pharmaceutical mixture may contain less than 100 mg/mL of the therapeutic agent and excipient. The pharmaceutical mixture may contain less than 100, 90, 80, 70, 60, 50, 40, 30, 20, 17.5, 15, 12.5, 10, 7.5, 5, 2.5, or 1 mg/mL, or any range derivable therein. Alternatively, the pharmaceutical mixture may comprise less than 10% w/v of the active pharmaceutical ingredient and excipient. The solid content or amount of active pharmaceutical ingredient and excipient in the pharmaceutical mixture is less than 10% w/v, less than 7.5% w/v, less than 5% w/v, less than 4% w/v, less than 3% w/v, less than 2.5% w/v, less than 2% w/v, less than 1.5% w/v, less than 1% w/v, or less than 0.5% w/v. In some embodiments, the solid content is from about 0.05% w/v to about 10% w/v, from about 0.1% w/v to about 5% w/v, from about 0.2% w/v to about 2.5% w/v, or from about 0.25% w/v to about 1% w/v. In some embodiments, the solid content is from about 0.05% w/v, 0.1% w/v, 0.15% w/v, 0.2% w/v, 0.25% w/v, 0.3% w/v, 0.4% w/v, 0.5% w/v, 0.6% w/v, 0.7% w/v, 0.8% w/v, 0.9% w/v, 1% w/v, 1.5% w/v, 2% w/v, 2.5% w/v, 3% w/v, 4% w/v, 5% w/v, to about 10% w/v, or any range derivable therein.

This pharmaceutical mixture may be deposited on a surface which is at a temperature that causes the pharmaceutical mixture to freeze. In some embodiments, this temperature may be below the freezing point of the solution at ambient pressure. In some embodiments, the pharmaceutical mixture is applied to a surface which has a temperature such as a processing temperature less than about 0° C. The processing temperature may be from about 0° C. to about −190° C., from about −25° C. to about −150° C., or from about −50° C. to about −125° C. The processing temperature may be from about 0° C., −25° C., −50° C., −60° C., −70° C., −80° C., −85° C., −90° C., −95° C., −100° C., −105° C., −110° C., −115° C., −120° C., −130° C., −140° C., −150° C., −160° C., −180° C., to about −190° C., or any range derivable therein. In other embodiments, a reduced pressure may be applied to the surface causing the solution to freeze at a temperature below the ambient pressure's freezing point. The pharmaceutical mixture may be applied from a height from about 1 cm to about 100 cm, from about 2 cm to about 50 cm, from about 5 cm to about 20 cm, or from about 5 cm to about 15 cm. The height may be from about 1 cm, 2 cm, 3 cm, 5 cm, 6 cm, 7 cm, 8 cm, 9 cm, 10 cm, 11 cm, 12 cm, 13 cm, 14 cm, 15 cm, 17 cm, 19 cm, 20 cm, 22 cm, 25 cm, 30 cm, 35 cm, 40 cm, 45 cm, to about 50 cm, or any range derivable therein. The pharmaceutical mixture may be applied to the surface using a nozzle such as a needle, pipette, small tubing, untrasonic nozzle, or funnel. The nozzle may contain one or more liquid outputs such as two, three, or four nozzles. The application of the pharmaceutical mixture to the surface may occur at a feed rate from about 0.5 mL/min to about 50 mL/min, from about 0.5 mL/min to about 10 mL/min, or from about 1 mL/min to about 3 mL/min The feed rate of the methods used herein may be from about 0.25 mL/min, 0.5 mL/min, 0.75 mL/min, 1 mL/min, 1.25 mL/min, 1.5 mL/min, 1.6 mL/min, 1.7 mL/min, 1.8 mL/min, 1.9 mL/min, 2 mL/min, 2.1 mL/min, 2.2 mL/min, 2.3 mL/min, 2.4 mL/min, 2.5 mL/min, 2.75 mL/min, 3 mL/min, 3.25 mL/min, 3.5 mL/min, 3.75 mL/min, 4 mL/min, 5 mL/min, 6 mL/min, 7 mL/min, 8 mL/min, 9 mL/min, 10 mL/min, 20 mL/min, 25 mL/min, 30 mL/min, 40 mL/min, to about 50 mL/min, or any range derivable therein.

The surface may also be rotating or moving on a moving conveyer-type system thus allowing the pharmaceutical mixture to distribute evenly on the surface. Alternatively, the pharmaceutical mixture may be applied to surface in such a manner to generate an even surface. The surface may be rotating at a speed of at least 5 rpm, at least 10 rpm, at least 25 rpm, or at least 50 rpm. The surface may be rotating at a speed from about 5 rpm to about 500 rpm, from about 100 rpm to about 400 rpm, or from about 150 rpm to about 250 rpm. The surface may be rotating from about 5 rpm, 10 rpm, 25 rpm, 50 rpm, 75 rpm, 100 rpm, 125 rpm, 150 rpm, 160 rpm, 170 rpm, 180 rpm, 190 rpm, 200 rpm, 210 rpm, 220 rpm, 230 rpm, 240 rpm, 250 rpm, 275 rpm, 300 rpm, 325 rpm, 350 rpm, 400 rpm, 450 rpm, to about 500 rpm, or any range derivable therein.

After the pharmaceutical mixture has been applied to the surface and collected, the solvent may be removed to obtain a pharmaceutical composition. Any appropriate method of removing the solvent may be applied including evaporation under reduced pressure or elevated temperature or lyophilization. In some embodiments, the lyophilization may comprise a reduced pressure and/or a reduced temperature. Such a reduced temperature may be from 25° C. to about −200° C., from 20° C. to about −175° C., from about 20° C. to about −150° C., from 0° C. to about −125° C., from −20° C. to about −100° C., from −75° C. to about −175° C., or from −100° C. to about −160° C. The temperature is from about −20° C., −30° C., −35° C., −40° C., −45° C., −50° C., −55° C., −60° C., −70° C., −80° C., −90° C., −100° C., −110° C., −120° C., −130° C., −140° C., −150° C., −160° C., −170° C., −180° C., −190° C., to about −200° C., or any range derivable therein. Additionally, the solvent may be removed at a reduced pressure of less than 500 mTorr, 450 mTorr, 400 mTorr, 375 mTorr, 350 mTorr, 325 mTorr, 300 mTorr, 275 mTorr, 250 mTorr, 225 mTorr, 200 mTorr, 175 mTorr, 150 mTorr, 125 mTorr, 100 mTorr, 75 mTorr, 50 mTorr, or 25 mTorr, or removed at a reduced pressure at any range of pressures derivable therein.

Such as composition prepared using these methods may exhibit a brittle nature such that the composition is easily sheared into smaller particles when processed through a device. These compositions have a high skeletal density and have high surface areas as well as exhibit improved flowability of the composition relative to compounds which are processed through other methods. Such flowability may be measured, for example, by the Carr index or other similar measurements. In particular, the Carr index may be measured by comparing the bulk density of the powder with the tapped density of the powder. Such compounds may exhibit a favorable Carr index and may result in the particles being better sheared to give smaller particles when the composition is processed through a secondary device to deliver the drug.

### III. DEFINITIONS

The use of the word “a” or “an” when used in conjunction with the term “comprising” in the claims and/or the specification may mean “one,” but it is also consistent with the meaning of “one or more,” “at least one,” and “one or more than one.” As used herein “another” may mean at least a second or more.

As used herein, the terms “active pharmaceutical ingredient”, “drug”, “pharmaceutical”, “active agent”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

The terms “compositions,” “pharmaceutical compositions,” “formulations,” “pharmaceutical formulations,” “preparations”, and “pharmaceutical preparations” are used synonymously and interchangeably herein.

“Treating” or treatment of a disease or condition refers to executing a protocol, which may include administering one or more drugs through one or more routes of administration to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of viral infection may involve, for example, a reduction of viral load, elimination of one or more symptoms, or complete elimination of the viral infection. Treatment of a viral infection may also refer to prolonging survival of a subject with cancer.

“Subject” and “patient” refer to either a human or non-human, such as primates, mammals, and vertebrates. In particular embodiments, the subject is a human.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic 30 acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

“Prodrug” means a compound that is convertible in vivo metabolically into an active pharmaceutical ingredient of the present invention. The prodrug itself may or may not have activity in its prodrug form. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

The term “derivative thereof” refers to any chemically modified polysaccharide, wherein at least one of the monomeric saccharide units is modified by substitution of atoms or molecular groups or bonds. In one embodiment, a derivative thereof is a salt thereof. Salts are, for example, salts with suitable mineral acids, such as hydrohalic acids, sulfuric acid or phosphoric acid, for example hydrochlorides, hydrobromides, sulfates, hydrogen sulfates or phosphates, salts with suitable carboxylic acids, such as optionally hydroxylated lower alkanoic acids, for example acetic acid, glycolic acid, propionic acid, lactic acid or pivalic acid, optionally hydroxylated and/or oxo-substituted lower alkanedicarboxylic acids, for example oxalic acid, succinic acid, fumaric acid, maleic acid, tartaric acid, citric acid, pyruvic acid, malic acid, ascorbic acid, and also with aromatic, heteroaromatic or araliphatic carboxylic acids, such as benzoic acid, nicotinic acid or mandelic acid, and salts with suitable aliphatic or aromatic sulfonic acids or N-substituted sulfamic acids, for example methanesulfonates, benzenesulfonates, p-toluenesulfonates or N-cyclohexylsulfamates (cyclamates).

The term “dissolution” as used herein refers to a process by which a solid substance, here the active ingredients, is dispersed in molecular form in a medium. The dissolution rate of the active ingredients of the pharmaceutical dose of the invention is defined by the amount of drug substance that goes in solution per unit time under standardized conditions of liquid/solid interface, temperature and solvent composition.

As used herein, the term “aerosols” refers to dispersions in air of solid or liquid particles, of fine enough particle size and consequent low settling velocities to have relative airborne stability (See Knight, V., Viral and Mycoplasmal Infections of the Respiratory Tract. 1973, Lea and Febiger, Phila. Pa., pp. 2).

As used herein, “inhalation” or “pulmonary inhalation” is used to refer to administration of pharmaceutical preparations by inhalation so that they reach the lungs and in particular embodiments the alveolar regions of the lung. Typically, inhalation is through the mouth, but in alternative embodiments in can entail inhalation through the nose.

As used herein, “dry powder” refers to a fine particulate composition that is not suspended or dissolved in an aqueous liquid.

A “simple dry powder inhaler” refers a device for the delivery of medication to the respiratory tract, in which the medication is delivered as a dry powder in a single-use, single-dose manner In particular aspects, a simple dry powder inhaler has fewer than 10 working parts. In some aspects, the simple dry powder inhaler is a passive inhaler such that the dispersion energy is provided by the patient's inhalation force rather than through the application of an external energy source.

A “median particle diameter” refers to the geometric diameter as measured by laser diffraction or image analysis. In some aspects, at least either 50% or 80% of the particles by volume are in the median particle diameter range.

A “Mass Median Aerodynamic Diameter (MMAD)” refers to the aerodynamic diameter (different than the geometric diameter). The mass median aerodynamic diameter is the diameter at which 50% of the particles by mass are larger and 50% are smaller. This value can be calculated as described in United States Pharmacopeia Chapter 601 by plotting, on log probability paper, the percentages of mass less than the stated aerodynamic diameters versus the aerodynamic diameters. The MMAD is the intersection of the line with the 50% cumulative percent. Computational methods are often used.

An “emitted dose” refers to the mass of drug emitted per actuation that is actually available for inhalation at the mouth. The “fine particle fraction, % recovered” refers the mass of particles less than 5 μm in size divided by the total mass recovered after actuation. The “fine particle fraction, % delivered” refers the mass of particles less than 5 μm in size divided by the total emitted dose.

A “geometric standard deviation” refers to the dispersion of particle diameter and is defined as the ratio of the median diameter to the diameter at ±1 sd (σ) from the median diameter. In a cumulative distribution plot of the aerodynamic diameter and mass of particles, the GSD is calculated as the ratio of the median diameter to the diameter at 15.9% of the probability scale, or the ratio of the diameter at 84.1% on the probability scale to the median diameter using GSD=(d₈₄/d₁₆)^{1/2} where d₈₄ and d₁₆ represent the diameters at which 84% and 16% of the aerosol mass are contained, respectively, in diameters less than these diameters. These parameters may be calculated using the methods described in Laube, et al., 2011.

The term “amorphous” refers to a noncrystalline solid wherein the molecules are not organized in a definite lattice pattern. Alternatively, the term “crystalline” refers to a solid wherein the molecules in the solid have a definite lattice pattern. Alternatively, compositions may be semi-crystalline or contain mixtures of both amorphous or crystalline regions. The crystallinity of the active agent in the composition is measured by powder x-ray diffraction.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significant such as “significantly”) is not meant to imply statistical differences between two values but only to imply importance or the scope of difference of the parameter.

Throughout this application, the term “about” is used to indicate that a value includes the inherent variation of error for the device, the method being employed to determine the value, or the variation that exists among the study subjects or experimental studies. Unless another definition is applicable, the term “about” refers to ±5% of the indicated value.

As used herein, the term “substantially free of” or “substantially free” in terms of a specified component, is used herein to mean that none of the specified component has been purposefully formulated into a composition and/or is present only as a contaminant or in trace amounts. The total amount of all contaminants, by-products, and other material is present in that composition in an amount less than 2%. The term “essentially free of” or “essentially free” is used to represent that the composition contains less than 1% of the specific component. The term “entirely free of” or “entirely free” contains less than 0.1% of the specific component.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements and parameters.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### IV. EXAMPLES

To facilitate a better understanding of the present disclosure, the following examples of specific embodiments are given. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. In no way should the following examples be read to limit or define the entire scope of the disclosure.

### Example 1—Preparation of Remdesivir Dry Powder Formulations

### I. Materials and Methods

### A. Materials

The remdesivir for formulation was purchased from Medkoo Biosciences (Research Triangle Park, N.C., USA). Remdesivir, GS-441524, and its heavy isotope internal standards were purchased from Alsachim (Illkirch-Graffenstaden, France). Lactose monohydrate, leucine, polysorbate 20, acetonitrile (HPLC grade), and trifluoracetic acid were purchased from Fisher Scientific (Pittsburgh, Pa., USA). D-Mannitol was bought from Acros Organic (Fair lawn, N.J., USA). Dipalmitoylphosphotidylcholine (DPPC) was purchased from Avanti Polar Lipid, Inc. (Alabaster, Ala., USA). Cholesterol, albumin, transferrin, ascorbic acid and Hanks' Balanced Salt solution (HBBS) were purchased from Sigma-Aldrich (St. Louis, Mo., USA). Sulfobutylether-beta-cyclodextrin (SBECD, Captisol®) was kindly provided by CyDex Pharmaceuticals, Inc. High-resistance Monodose RS00 dry powder inhalers were kindly provided by Plastiape S.p.A. (Osnago, Italy).

### B. Preparation of Dry Powder for Inhalation Using Thin Film Freezing

Remdesivir and excipients (i.e., Captisol®, mannitol, lactose and leucine) were dissolved in a mixture of either acetonitrile/water (50/50 v/v) or 1,4-dioxane/water (50/50 v/v) at the solid contents shown in Table 1. The solutions were stored in a refrigerator at 2-8° C. before the thin film freezing process. The solution was passed through an 18-gauge syringe needle and dropped from a height of approximately 10 cm onto a rotating cryogenically cooled stainless-steel drum. The solutions were frozen at a drum surface temperature of −100° C. The frozen samples were collected in a stainless-steel container filled with liquid nitrogen and then transferred into a −80° C. freezer (Thermo Fisher Scientific, Waltham, Mass., USA) before drying in a lyophilizer (SP Industries Inc., Warminster, Pa., USA). The frozen samples were primary dried at −40° C. for 20 h, and ramped to 25° C. over 20 h, and then secondary dried at 25° C. for 20 h. The vacuum pressure was set at 100 mTorr for the whole drying cycle.

TABLE 1
List of thin film freezing (TFF) remdesivir
compositions and parameters used for their preparation.

| | | | | | Processing |
|---|---|---|---|---|---|
| | Drug | | Solid | | Temper- |
| Formu- | Loading | | Content | Solvent | ature |
| lation | (% w/w) | Excipient | (% w/v) | (v/v) | (° C.) |
| F1 | 5 | Captisol ® | 1.00 | Acetonitrile/water | −100 |
| | | | | (50/50) | |
| F2 | 20 | Captisol ® | 1.00 | Acetonitrile/water | −100 |
| | | | | (50/50) | |
| F3 | 20 | Mannitol | 1.00 | Acetonitrile/water | −100 |
| | | | | (50/50) | |
| F4 | 20 | Lactose | 1.00 | Acetonitrile/water | −100 |
| | | | | (50/50) | |
| F5 | 20 | Leucine | 0.75 | Acetonitrile/water | −100 |
| | | | | (50/50) | |
| F6 | 50 | Captisol ® | 0.30 | Acetonitrile/water | −100 |
| | | | | (50/50) | |
| F7 | 50 | Mannitol | 0.30 | Acetonitrile/water | −100 |
| | | | | (50/50) | |
| F8 | 50 | Lactose | 0.30 | Acetonitrile/water | −100 |
| | | | | (50/50) | |
| F9 | 50 | Leucine | 0.30 | Acetonitrile/water | −100 |
| | | | | (50/50) | |
| F10 | 80 | Captisol ® | 0.30 | Acetonitrile/water | −100 |
| | | | | (50/50) | |
| F11 | 80 | Mannitol | 0.30 | Acetonitrile/water | −100 |
| | | | | (50/50) | |
| F12 | 80 | Lactose | 0.30 | Acetonitrile/water | −100 |
| | | | | (50/50) | |
| F13 | 80 | Leucine | 0.30 | Acetonitrile/water | −100 |
| | | | | (50/50) | |
| F14 | 100 | — | 0.25 | Acetonitrile/water | −100 |
| | | | | (50/50) | |
| F15 | 100 | — | 0.50 | 1,4-dioxane/water | −100 |
| | | | | (50/50) | |
| F16 | 100 | — | 1.00 | 1,4-dioxane/water | −100 |
| | | | | (50/50) | |
| F17 | 80 | Captisol ® | 1.00 | 1,4-dioxane/water | −100 |
| | | | | (50/50) | |
| F18 | 80 | Mannitol | 1.00 | 1,4-dioxane/water | −100 |
| | | | | (50/50) | |
| F19 | 80 | Lactose | 1.00 | 1,4-dioxane/water | −100 |
| | | | | (50/50) | |
| F20 | 80 | Leucine | 1.00 | 1,4-dioxane/water | −100 |
| | | | | (50/50) | |

### C. Drug Quantification (HPLC)

The content of remdesivir was determined through analysis with a Thermo Scientific™ Dionex™ UltiMate™ 3000 HPLC system (Thermo Scientific, Sunnyvale, Calif., USA) at a wavelength of 246 nm. A Waters Xbridge C18 column (4.6×150 mm, 3.5 μm) (Milford, Mass., USA) was used at 30° C. and a flow rate of 0.8 mL/min. The isocratic method was performed for 4.5 min using a mobile phase of 50:50 (% v/v) water-acetonitrile containing 0.05% (v/v) TFA. The retention time of remdesivir was approximately ˜3.5 min Dimethyl sulfloxide: acetonitrile: water (10:60:30, v/v) was used as diluent. All analyses exhibited linearity in the range tested of 0.2-250 μg/mL. All chromatography data were processed by Chromeleon Version 7.2.10 software (Thermo Fisher Scientific, Waltham, Mass., USA).

For quantity and quality analysis of stability samples of TFF remdesivir powders, the Agilent 1220 Infinity II LC system (Agilent Technologies, Inc., Santa Clara, Calif., USA) equipped with an Agilent 1290 Infinity II evaporative light scattering detector (ELSD) (Agilent Technologies, Inc., Santa Clara, Calif., USA) was utilized. Waters XBridge BEH C18 column (4.6×150 mm, 3.5 μm) (Waters Corporation, Milford, Mass., USA) was used at 30° C. and a flow rate of 0.8 mL/min The gradient method with a mobile phase ratio of 5 to 95% acetonitrile in water with 0.1% trifluoroacetic acid was used for total running time of 20 min. In total, 10 μL of each sample was injected. The stability samples were monitored with 246 nm UV and ELSD to detect possible degradants. For ELSD, evaporator and nebulizer temperatures were set at 60° C., and nitrogen gas flow was 1.6 L/min. The chromatography data were processed by Chemstation version C.01.10 (Agilent Technologies, Inc., Santa Clara, Calif., USA).

### D. In Vitro Aerosol Performance

The aerodynamic properties of the TFF powder samples were determined using a Next Generation Pharmaceutical Impactor (NGI) (MSP Corp, Shoreview, Minn., USA) connected to a High-Capacity Pump (model HCPS, Copley Scientific, Nottingham, UK) and a Critical Flow Controller (model TPK 2000, Copley Scientific, Nottingham, UK). The pre-separator was not used in this study. To avoid particle bounce, the NGI collection plates were coated with 1.5% w/v polysorbate 20 in 100% methanol and then let the plates dry before analysis. A high-resistance Plastiape® RS00 inhaler (Plastiape S.p.A, Osnago, Italy) containing size #3 hydroxypropyl methylcellulose capsules (V-Caps® Plus, Lonza, Morristown, N.J.) and 1-3 mg of powder was attached to a USP induction port by a molded silicon adapter, and the powder was dispersed to the NGI at the flow rate of 60 L/min for 4 s per each actuation, providing a 4 kPa pressure drop across the device. The deposited powders from the capsule, inhaler, adapter, induction port, stages 1-7, and the micro-orifice collector (MOC) were collected by diluting with a mixture of dimethyl sulfoxide (DMSO)/water/acetonitrile (10:30:60 v/v/v). The remdesivir content in the deposited powders was determined using the HPLC method.

Copley Inhaler Testing Data Analysis Software (CITDAS) Version 3.10 (Copley Scientific, Nottingham, UK) was used to calculate the mass median aerodynamic diameter (MMAD), the geometric standard deviation (GSD), the fine particle fraction (FPF), and the emitted fraction (EF). The EF was calculated as the total amount of remdesivir emitted from the device as a percentage of total amount of remdesivir collected. The FPF of recovered dose was calculated as the total amount of remdesivir collected with an aerodynamic diameter below 5 μm or 3 μm as a percentage of the total amount of remdesivir collected. The FPF of delivered dose was calculated as the total amount of remdesivir collected with an aerodynamic diameter below 5 μm or 3 μm as a percentage of the total amount of remdesivir deposited on the adapter, the induction port, stages 1-7 and MOC.

### E. X-Ray Powder Diffraction (XRPD)

The crystallinity of the powders was evaluated using a benchtop X-ray diffraction instrument (Rigaku Miniflex 600 II, Woodlands, Tex., USA) equipped with primary monochromated radiation (Cu K radiation source, λ=1.54056 Å). The instrument was operated at an accelerating voltage of 40 kV at 15 mA. Samples were loaded in the sample holder and scanned in continuous mode with a step size of 0.02° over a 2θ range of 5-40° at a scan speed of 2°/min, and a dwell time of 2 s.

### F. Modulated Differential Scanning Calorimetry (mDSC)

Thermal analysis of powder samples was conducted using a differential scanning calorimeter Model Q20 (TA Instruments Inc., New Castle, Del., USA) equipped with a refrigerated cooling system (RCS40, TA Instruments Inc., New Castle, Del., USA). Samples of 2-3 mg were weighed and loaded into a T-zero pan. The pan with a T-zero hermetic lid were crimped, and a hole was drilled in the lid before placing the pan in the sample holder. To evaluate the glass transition and glass-forming ability type of remdesivir unprocessed powder, samples were heat at a heating ramp rate of 10° C./min from 25° C. to 150° C., then cooled down to −40° C., and then heated again to 250° C. To investigate the crystallinity of the TFF formulations, samples were heated from 25° C. to 350° C. with a heating ramp rate of 5° C./min The scans were performed with a modulation period of 60 s and a modulated amplitude of 1° C. Dry nitrogen gas at a flow rate of 50 mL/min was used to purge the DSC cell throughout the analysis. Data were processed by TA Instruments Trios V.5.1.1.46572 software (TA Instruments, Inc., New castle, Del., USA).

### G. Scanning Electron Microscopy (SEM)

Scanning electron microscopy (Zeiss Supra 40 C SEM, Carl Zeiss, Heidenheim an der Brenz, Germany) was used to identify surface particle morphology of the TFF remdesivir powder formulations. A small amount from each of the powder formulations was placed on a carbon tape. A sputter coater was used to coat all samples with 15 nm of 60/40 Pd/Pt before capturing the images.

### H. Nuclear Magnetic Resonance (NMR)

¹H-NMR spectra were obtained using a Varian® NMR 400 MHz Spectrometer (Agilent Inc., Palo Alto, Calif., USA) at 25° C. and were used to confirm the purity of remdesivir in the TFF powders, and to study interactions between excipients and remdesivir of TFF powders. Formulations F7, F8, F9, F10, F14 and unprocessed L-leucine and remdesivir drug substance were dissolved in dimethyl sulfoxide-d6 (DMSO-d6). Solutions were then transferred to 5 mm NMR tubes for NMR data acquisition. Chemical shifts were referenced to a residual solvent, DMSO, at 2.50 ppm. The NMR spectrum peaks of remdesivir in the formulations produced by TFF were compared to those of remdesivir unprocessed powder as received from the vendor. Excipients peaks in TFF remdesivir powders were also compared to those of unprocessed excipients.

### I. Preparation of Simulated Lung Fluid (SLF)

The composition and preparation of simulated lung fluid (SLF) were adapted from Hassoun's study (Hassoun et al., 2018). SLF contained dipalmitoylphosphotidylcholine (DPPC), cholesterol, albumin, transferrin, ascorbic acid and Hank's balanced salt solution (HBSS). The SLF was prepared by two steps including a preparation of liposomal dispersion and freeze drying as described in Hassoun et al. (Hassoun et al., 2018). In the first step, a liposomal dispersion was prepared by mixing 2.12 mL of 25 mg/mL DPPC and 5 μL of 200 mg/mL cholesterol in chloroform. Then, the solution was dried using an evaporator until the dry film was obtained. The thin film of lipid was dispersed by adding 8 mL of 57 mg/mL bovine serum albumin in water, 1 mL of 15 mg/mL transferrin in water, and 88.5 μL of 10 mM ascorbate solution. The mixture was sonicated until it fully dispersed. The dispersion was adjusted to 10 mL by adding 1M HBSS). The dispersion was frozen at −20° C. for 6 h, and primary dried in the lyophilizer at −40° C. for 30 h and ramp to 25° C. for 12 h and finally secondary dried at 25° C. for 20 h. The lyophilized SLF was stored at 2-8° C. and reconstituted with purified water upon solubility and dissolution testing.

### J. Solubility Testing

The solubility of crystalline remdesivir, amorphous remdesivir, F10 and F13 in SLF was evaluated. The excess amount of powder was added in SLF until precipitation occurred. Then the samples were shaken on the orbital shaker at 250 rpm and 25° C. for 24 h. The samples were collected and centrifuged at 13,000 rpm for 15 min. The supernatants were collected and then diluted with DMSO/acetonitrile (10/90). The samples were centrifuged again at 13,000 rpm for 15 min to separate proteins in SLF. The supernatants were collected and analyzed by HPLC.

### K. Dissolution Testing

Dissolution testing of F10, F11, F12, F13, F14 and unprocessed crystalline remdesivir was performed under sink conditions in SLF and using a transwell system based upon previously published studies (Arora et al., 2010; Brunaugh et al., 2020; Rohrschneider et al., 2015). An NGI (MSP Corp, Shoreview, Minn., USA) connected to a High-Capacity Pump (model HCPS, Copley Scientific, Nottingham, UK) and a Critical Flow Controller (model TPK 2000, Copley Scientific, Nottingham, UK) was used to load aerosolized powder. Three 24-mm diameter filter papers were placed on stage 4 on the NGI to capture aerosolized powder, which corresponded to an aerodynamic size cut off of 1.66 μm at 60 L/min The transwell systems were modified as described in Rohrschneider et al. (Rohrschneider et al., 2015). The filter papers were loaded in the donor compartment of the modified transwell systems. Reconstituted SLF was heated up to 37° C. before loading 1.5 mL in the six-well plate. The experiment was initiated after placing the donor compartment in the plate and adding 0.1 mL of SLF onto the filter papers. The modified transwell systems was placed on an orbital shaker at 100 rpm and 37° C. Samples of 0.1 mL were collected at 10, 20, 30, 45, 60, 90, 120, 180, 240, 360, and 540 min. Samples were diluted with DMSO/acetonitrile (10/90) and centrifuged at 13,000 rpm for 15 min to separate proteins in SLF. Supernatants were collected and analyzed by HPLC, as described in Section 2.3. Removed volumes were refilled with fresh SLF to maintain the dissolution volume. At the last time point, the filter membranes were removed and washed with DMSO/acetonitrile (10/90) to analyze the amount of undissolved drug.

### L. Stability Study

The TFF powder of F10, F12 and F13 was transferred to a borosilicate glass vial. The vials were dried in a lyophilizer at 25° C. and 100 mTorr for 6 h, then backfilled with nitrogen gas before stoppering inside the lyophilizer. The vials were sealed with aluminum caps before packing in an aluminum foil bag with a desiccant. All samples were stored at 25° C./60% relative humidity (RH). After one month, samples were collected and analyzed to determine their physical stability and chemical stability. Additionally, samples were analyzed to determine aerosol performance.

### M. In Vivo Pharmacokinetic Study

### i. Single-Dose Dry Powder Insufflation with Sprague-Dawley Rats

An in vivo pharmacokinetic study was conducted using Sprague-Dawley rats in compliance with the Institutional Animal Care and Use Committee (IACUC; Protocol Number AUP-2019-00253) guidelines at The University of Texas at Austin. Rats weighing between 200 and 250 g (average weight of 214.3 g) were housed in a 12-h light/dark cycle with access to food and water ad libitum and were subjected to one week of acclimation time to the housing environment.

Intratracheal administration was carried out using the dry powder insufflator (DP-4 model, Penn-Century Inc., Philadelphia, Pa., USA) connected to the air pump (AP-1 model, Penn-Century Inc., Philadelphia, Pa., USA). TFF powder was passed through a No. 325 sieve (45 μm aperture) to break down large aggregates. TFF remdesivir F10 and F13 were given to 5 rats per group. A precisely weighed quantity of sieved TFF powder was introduced into the sample chamber of the insufflator device. The dose was targeted at 10 mg/kg. Each rat was placed on its back at a 45° angle, and its incisors were secured with a rubber band. A laryngoscope was used to visualize the trachea and insert the insufflator device into the trachea. The sieved TFF powder was actuated into the lung using 3-5 pumps (200 μL of air per pump). The mass of powder delivered was measured by weighing the chamber before and after dose actuations.

Following insufflation, whole blood was collected at each time point (5, 15, 30, 2, 4, 8, and 24 h) by cardiac puncture into heparinized vials. The blood samples were centrifuged at 10,000 rpm for 3 min to obtain plasma. Lung were harvested after collecting blood at the last time point. Plasma samples and lungs were frozen and stored at −20° C. until analysis.

### ii. Single-Dose Dry Powder Insufflation with Syrian Hamsters

This study was in compliance with the Institutional Animal Care and Use Committee (IACUC; Protocol Number AUP-2019-00254) guidelines at The University of Texas at Austin. Female Syrian hamsters (Charles River, 049LVG) 35-42-days old and weighing between 80 and 130 g (average weight of 102 g) were housed in a 12-hour light/dark cycle with access to food and water ad libitum and were subjected to one week of acclimation to the housing environment. Seventy hamsters were separated into two equal groups by body weight (REM-CAP and REM-LEU).

TFF powder formulation was passed through a No. 200 sieve (75 μm aperture) to break down large aggregates into fine particles. Precisely weighed quantities of sieved TFF powder were administered to hamsters intratracheally using a dry powder insufflator (DP-4 model, Penn-Century Inc., Philadelphia, Pa., USA) connected to an air pump (AP-1 model, Penn-Century Inc., Philadelphia, Pa., USA). The dose of remdesivir was targeted to be 10 mg/kg. Each hamster was briefly anesthetized with isoflurane (4% induction, 2% maintenance) and placed on its back on an intubation stand. Its upper incisors were used to secure the hamster using silk at a 45° angle, with continuous delivery of anesthesia through a nose cone. A laryngoscope was used to visualize the trachea, and the blunt metal end of the insufflator device was inserted into the trachea. The sieved TFF powder was actuated into the lung using 3 puffs of the connected pump (200 μL of air per puff). The mass of powder delivered was measured by weighing the device chamber before and after dose actuations.

Following powder administration, five hamsters from each group were harvested at each time point (15 mins, 30 mins, 1, 2, 4, 8, 24 hours). Blood was drawn via cardiac puncture and immediately transferred into a heparinized microtainer (BD, 365985, Lithium Heparin/PST™ Gel). The blood sample was centrifuged at 10,000 rpm for 1.5 minutes, and the plasma was separated and frozen on dry ice. The hamster was carefully perfused with PBS, the lung was washed with 1 mL of PBS to remove the residual dry powder, and the lung was removed, weighed and frozen. Plasma samples and lungs were kept frozen and stored at −80° C. until analysis.

### iii. Analysis of Remdesivir and Its Metabolites in Plasma and Lung Tissue

Plasma samples were prepared by combining 100 μL of plasma with 100 μL of methanol that contained 100 ng/mL of the heavy labeled internal standards for remdesivir and GS-441524, followed by vortexing and centrifuging at 12,000 rpm for 15 min. The supernatant was placed in a 96-well plate for LC/MS/MS analysis.

Lung tissue samples were cut in small pieces, added into a 2 mL tube with 3.5 g of 2.3 mm zirconia/silica beads (BioSpec Producs, Bartlesville, Okla., USA), and homogenized at 4800 rpm for 20 s. After homogenization, 1000 μL of methanol, containing 100 ng/mL internal standards for remdesivir and GS-441524 was added to the tube, and the tube was vortexed and centrifuged at 12,000 rpm for 15 min. The supernatant was placed in a 96-well plate for LC/MS/MS analysis. Calibration standards were prepared for plasma and lung tissue in the same fashion, spiking remdesivir and GS-441524 standard solutions into the blank plasma and lung tissue to obtain matrix matched calibrations. The calibration range was 0.1-1000 ng/mL for plasma and 50-10,000 ng/mL for lung tissue. The calibration range was chosen to bracket sample levels measured.

Remdesivir and GS-441524 were separated on an Agilent Poroshell column (2.1×50 mm, 2.7 μm) (Agilent, Santa Clara, Calif., USA) using a gradient of 0 to 90.25% of acetonitrile with 0.025% trifluoroacetic acid in 5 min at a flow rate of 0.35 mL/min, and a column temperature of 40° C. In total, 10 μL of each sample was injected for analysis with an Agilent 6470 triple quadrupole LC/MS/MS system (Agilent, Santa Clara, Calif., USA).

### iv. Pharmacokinetic Analysis

The concentration of remdesivir and GS-441524 in plasma versus time was plotted. The data are presented as the mean±standard deviation (SD). The plots were used to compare the pharmacokinetic behavior of F10 and F13. Microsoft Excel 2016 was used to perform a one compartmental analysis to determine key pharmacokinetic parameters including area under the plasma concentration-time curve from time 0 to 24 hours (AUC_{0-24 h}), and area under the plasma concentration-time curve from time 0 extrapolated to infinity (AUC_{inf}).

For the hamster studies, remdesivir and metabolite GS-441524 plasma and lung concentrations over time data were analyzed by non-compartmental analysis using PKSolver to obtain pharmacokinetic parameters in the hamster model after inhalation of the formulations (Watts et al., 2013). Due to the sparse sampling requirements with this animal model and to obtain lung concentrations over time a naïve pooled-data approach was used in which the noncompartmental analysis was fit to the data as if the average of the measured concentrations from the five animals at each time point were taken from a single subject. This was based on the methods previously reported for estimating population kinetics from very small sample sizes (Wang et al., 2014; Longest et al., 2017).

### N. Statistical Analysis

The statistical significance of experimental results was conducted using Student's t-test in JMP 10 (SAS Institute, Cary, N.C., USA). The alpha level was set at 0.05.

### II. Results

### A. Physical Properties of TFF Remdesivir

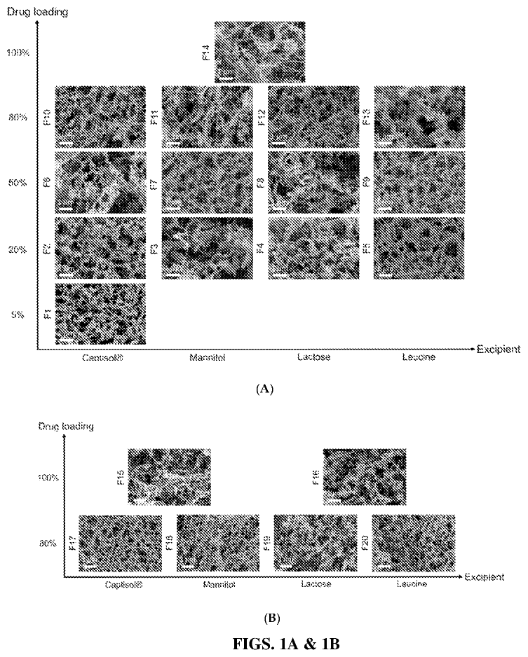

The particle morphology of TFF remdesivir powder formulations containing different drug loading concentrations and different excipients was determined using SEM. All formulations exhibited a brittle matrix structure of highly porous particles (FIG. 1). For acetonitrile/water solvent system, Captisol®- and leucine-based formulations containing the same drug loading showed relatively smaller nanostructure aggregates, compared to mannitol- and lactose-based formulations. Different cosolvent systems resulted in different brittle matrix powder morphologies as observed by SEM. At the same drug load, mixture prepared from 1,4-dioxane/water produced smaller nanostructure aggregates, compared to an equivalent mixture prepared from acetonitrile/water (FIG. 1B).

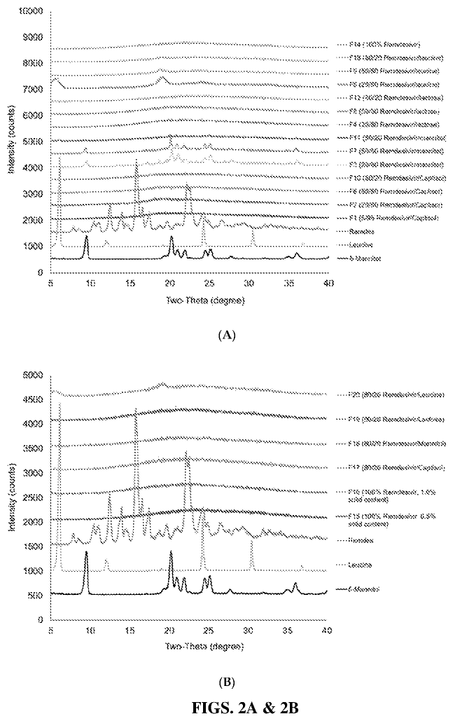

FIG. 2 shows the x-ray diffraction patterns of TFF remdesivir powder formulations. No sharp peaks of remdesivir were observed in any of formulations, indicating that remdesivir was amorphous after the TFF process. The drug loading and type of co-solvent did not affect the morphology of remdesivir. For excipients, sharp peaks of mannitol (13.5, 17, 18.5, 20.2,21, 22, 24.5, 25, 27.5, and 36 degree two-theta) were observed in F3, F7 and F11, indicating that mannitol in these formulations remained crystalline as a mixture of δ and α form (Cares-Pacheco et al., 2014). Similarly, some peaks of leucine (6 and 19 degree two-theta) were observed in the TFF remdesivir-leucine formulations, F5, F9, F13, and F20, indicating that leucine remained crystalline after the process. In contrast, Captisol® and lactose in the compositions were amorphous after the TFF process.

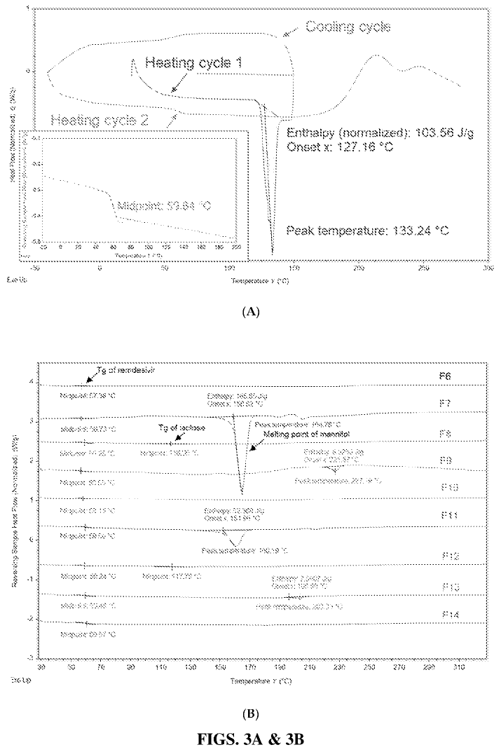

Modulated differential scanning calorimetry (mDSC) was employed to identify the glass-forming ability of remdesivir and determine the glass transition temperature (T_{g}) of remdesivir in each formulation. FIG. 3A shows a mDSC thermogram of remdesivir unprocessed powder. The first heating cycle demonstrated that remdesivir was crystalline with a melting point of ˜133° C. The cooling and second heating cycle showed that the glass transition temperature of remdesivir was about 60° C. No recrystallization peak was observed in any cycle.

FIG. 3B shows the physical state of remdesivir and excipients. Despite the different formulation compositions, no melting peak of remdesivir was observed in any formulations, and it remained amorphous. These results agree with XRD diffractograms showing that remdesivir was amorphous after the TFF process. The mDSC thermograms showed that the T_{g} of remdesivir in all formulations was in the range 56-61° C. Captisol® in both F6 and F9 showed no endothermic peaks, demonstrating that Captisol® was in amorphous form. Endothermic peak of mannitol in F7 and F11 was observed at ˜165° C. and ˜160° C., respectively, indicating mannitol was crystalline after TFF process. For lactose-based formulations, two glass transition temperatures were observed, including T_{g} of remdesivir (˜59° C.) and T_{g} of lactose (˜117° C.), indicating lactose and remdesivir were not molecularly miscible. For leucine-based formulations, an endothermic peak was observed in F9 and F13 at ˜227° C. and ˜203° C., respectively, which are not identical with the melting peak of unprocessed leucine (˜300° C.) (Molina et al., 2018). Therefore, the melting point depression of leucine can be attributed to an interaction of remdesivir and leucine.

### B. Aerodynamic Properties of TFF Remdesivir

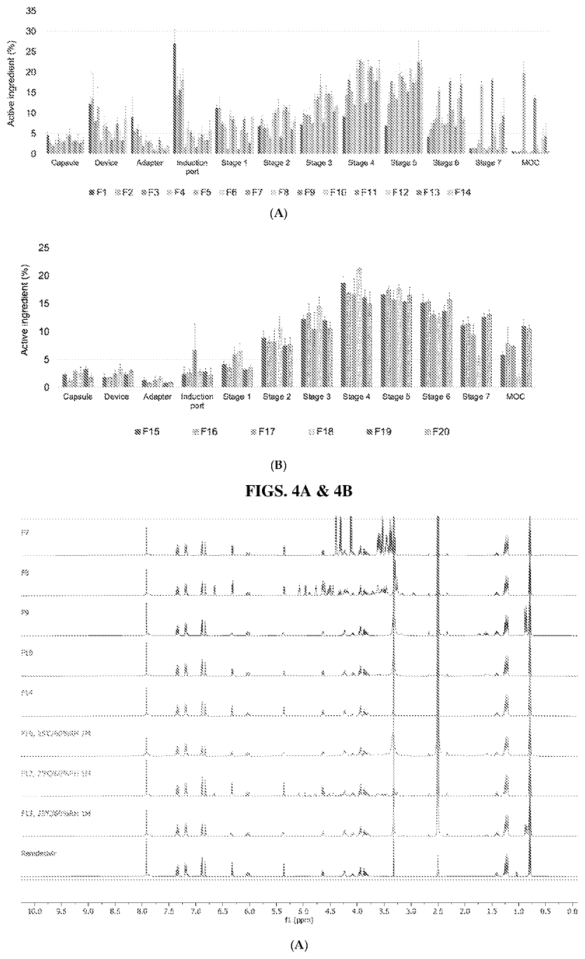

The aerodynamic particle size distribution of TFF remdesivir formulations was evaluated using a Plastiape® RS00 high-resistance DPI and NGI apparatus. FIG. 4 and Table 2 show aerodynamic diameter distribution and aerosol performance of TFF remdesivir formulations, respectively. In vitro aerodynamic testing revealed that drug loading affected the aerosol performance of the TFF formulations. Despite using different excipients, the aerosol performance generally increased as the drug loading was increased. The FPF_{<5 μm} (of the recovered dose) of Captisol®-based formulations containing 5%, 20%, 50% and 80% of remdesivir (F1, F2, F6, F10, respectively) were 30.73±1.11%, 45.84±6.03%, 65.19±1.18% and 68.29%, respectively. The MMAD of these TFF remdesivir powder formulations was 3.10±0.04 μm, 2.59±0.15 μm, 2.22±0.14 μm, and 2.16±0.21 μm, respectively. For lactose- and mannitol-based formulations, there were significant increases in FPF (of recovered dose) when the drug loading was increased from 20% to 50%; however, FPF did not significantly change when the drug loading was increased further from 50% to 80% with these materials. Interestingly, FPFs of leucine-based formulations containing 20%, 50% and 80% of remdesivir were not significantly different. However, the MMADs of these leucine-based formulations at drug loadings of 20%, 50% and 80% increased from 0.74 ±0.06 μm to 0.82±0.07 μm to 1.45±0.07 μm, respectively. When the drug loading was increased to 100% (excipient free), F14 exhibited an FPF_{<5 μm} of 62.33% (of recovered drug) and an MMAD of 2.06 μm.

An effect of the excipient on aerosol performance was also observed. At the same drug loading, leucine-based formulations exhibited significantly greater FPF (81-85% FPF_{<5 μm}), and a smaller MMAD (0.7-1.5 μm), compared to other excipients, indicating that leucine has distinct advantages over other compositions in terms of aerosol performance

The effect of co-solvent mixture and solid content on the aerosol performance of the formulations were also investigated. For 100% drug loading (no excipient present in composition), significant differences in MMAD, FPF_{<5 μm}, and EF were observed between the formulations prepared from the acetonitrile/water (F14) and 1,4-dioxane/water (F15 and F16) co-solvent systems. F15 and F16 exhibited a smaller MMAD, and a larger FPF and EF compared to F14. Comparing the formulations prepared in the same solvent system, F16 showed a better aerosol performance, although it was prepared at higher solid content.

Similar trends were observed in the Captisol®, mannitol, lactose, and leucine-based formulations. Captisol®, mannitol, lactose, and leucine-based formulations that were prepared in a 1,4-dioxane/water co-solvent (F17, F18, F19, F20, respectively) showed smaller MMAD, compared to the same compositions prepared in an acetonitrile/water cosolvent system (F10, F11, F12 and F13, respectively).

TABLE 2
Aerosol performance of TFF remdesivir powders using a Plastiape ® RS00 high-
resistance DPI at a flow rate of 60 L/min (n = 3). (MMAD, mass median aerodynamic
diameter; GSD, geometric standard deviation; FPF, fine particle fraction; EF, emitted fraction).

| | | | FPF_{<5 μm} | FPF_{<3 μm} | FPF_{<5 μm} | FPF_{<3 μm} | EF |
|---|---|---|---|---|---|---|---|
| | MMAD | | (%, Recovered | (%, Recovered | (%, Delivered | (%, Delivered | (%, Recovered |
| Formulations | (μm) | GSD | Dose) | Dose) | Dose) | Dose) | Dose) |
| F1 | 3.10 ± 0.04 | 2.88 ± 0.03 | 30.73 ± 1.11 | 22.16 ± 1.01 | 36.87 ± 0.78 | 27.78 ± 0.84 | 83.35 ± 1.83 |
| F2 | 2.59 ± 0.15 | 2.56 ± 0.28 | 45.84 ± 6.03 | 35.65 ± 5.97 | 55.00 ± 5.44 | 42.68 ± 4.88 | 83.32 ± 6.70 |
| F3 | 2.03 ± 0.10 | 2.76 ± 0.07 | 56.21 ± 2.16 | 46.69 ± 1.11 | 62.27 ± 2.72 | 51.72 ± 1.52 | 90.28 ± 0.67 |
| F4 | 2.02 ± 0.37 | 2.69 ± 0.10 | 52.38 ± 4.65 | 43.09 ± 6.64 | 60.98 ± 3.16 | 50.08 ± 5.50 | 85.86 ± 5.40 |
| F5 | 0.74 ± 0.06 | 3.50 ± 0.13 | 86.03 ± 2.82 | 78.64 ± 2.80 | 92.10 ± 1.76 | 84.19 ± 2.00 | 93.40 ± 1.73 |
| F6 | 2.22 ± 0.14 | 2.73 ± 0.22 | 65.19 ± 1.18 | 51.98 ± 2.16 | 72.17 ± 2.04 | 57.56 ± 3.11 | 90.37 ± 2.49 |
| F7 | 2.32 ± 0.07 | 2.57 ± 0.03 | 65.90 ± 0.91 | 51.65 ± 1.27 | 73.08 ± 0.22 | 57.28 ± 0.79 | 90.17 ± 0.97 |
| F8 | 2.40 ± 0.39 | 2.28 ± 0.11 | 68.43 ± 4.33 | 51.56 ± 7.23 | 75.74 ± 4.60 | 57.08 ± 7.92 | 90.34 ± 0.82 |
| F9 | 0.82 ± 0.07 | 3.19 ± 0.10 | 85.83 ± 3.96 | 78.32 ± 4.16 | 92.99 ± 1.11 | 84.84 ± 1.73 | 92.28 ± 3.23 |
| F10 | 2.16 ± 0.21 | 2.42 ± 0.06 | 71.48 ± 5.52 | 57.01 ± 6.25 | 78.08 ± 5.51 | 62.25 ± 5.65 | 91.48 ± 2.09 |
| F11 | 2.44 ± 0.06 | 2.53 ± 0.06 | 64.21 ± 3.53 | 48.90 ± 3.06 | 71.62 ± 3.10 | 54.54 ± 2.68 | 89.61 ± 1.30 |
| F12 | 2.03 ± 0.11 | 2.51 ± 0.12 | 70.32 ± 3.39 | 55.54 ± 2.70 | 77.24 ± 2.94 | 61.02 ± 2.85 | 91.02 ± 1.55 |
| F13 | 1.45 ± 0.07 | 2.17 ± 0.01 | 82.71 ± 2.54 | 73.34 ± 3.17 | 89.68 ± 0.91 | 79.51 ± 1.85 | 92.21 ± 1.94 |
| F14 | 2.09 ± 0.07 | 2.79 ± 0.13 | 65.80 ± 2.68 | 53.95 ± 1.26 | 74.44 ± 1.29 | 61.05 ± 0.43 | 88.37 ± 2.16 |
| F15 | 1.53 ± 0.16 | 2.70 ± 0.10 | 81.66 ± 0.80 | 69.08 ± 1.63 | 85.11 ± 1.20 | 72.00 ± 2.11 | 95.95 ± 0.56 |
| F16 | 1.42 ± 0.20 | 2.77 ± 0.18 | 84.25 ± 1.87 | 71.00 ± 3.91 | 86.77 ± 1.67 | 73.11 ± 3.86 | 97.10 ± 0.40 |
| F17 | 1.55 ± 0.16 | 2.99 ± 0.43 | 74.59 ± 7.03 | 63.74 ± 3.76 | 78.76 ± 6.75 | 67.30 ± 3.37 | 94.67 ± 0.97 |
| F18 | 2.03 ± 0.20 | 2.48 ± 0.07 | 75.34 ± 1.06 | 60.44 ± 3.13 | 79.94 ± 2.72 | 64.15 ± 4.60 | 94.30 ± 1.94 |
| F19 | 1.28 ± 0.10 | 2.92 ± 0.17 | 82.40 ± 1.18 | 70.49 ± 1.14 | 87.28 ± 0.57 | 74.55 ± 0.56 | 94.41 ± 0.83 |
| F20 | 1.29 ± 0.11 | 3.15 ± 0.18 | 82.26 ± 3.86 | 70.68 ± 4.39 | 86.09 ± 3.51 | 73.97 ± 4.15 | 95.53 ± 0.60 |

### C. Interactions Between TFF Remdesivir and Excipients

¹H-NMR was performed to identify interactions between remdesivir and excipients. FIG. 5B demonstrates an expansion of ¹H-NMR spectra for selected TFF remdesivir powder formulations and remdesivir unprocessed powder. While the peak at 6.03 ppm is sharp and does not show any differences from the presented samples, the peaks at 5.37 and 6.33 ppm of F9 were broader. Moreover, these peaks were slightly shifted downfield. FIG. 5C shows an expansion of the ¹H-NMR spectra of F9 and leucine unprocessed powder. The peak at 3.09 ppm of leucine was also shifted downfield to 3.13 ppm in F9. These results indicate that remdesivir and leucine form interactions during the TFF process.

### D. Stability Study of TFF Remdesivir

### i. Chemical Stability of TFF Remdesivir Compositions

The chemical stability of remdesivir after TFF processing was confirmed by ¹H-NMR. FIG. 5A presents the ¹H-NMR spectra of selected TFF remdesivir powder formulations and remdesivir unprocessed powder. Remdesivir peaks from the TFF remdesivir powder formulations consisting of 50% (w/w) remdesivir with mannitol, lactose, and leucine (F7, F8, and F9, respectively) are identical to the peaks of remdesivir unprocessed powder. The 80% (w/w) remdesivir with Captisol® (F10) and 100% remdesivir without excipient (F14) powders also presented indistinguishable peaks corresponding to remdesivir. Therefore, neither the aqueous-organic cosolvent mixture nor the conditions used in the TFF process affected the chemical stability of remdesivir.

After one month of storage at 25° C./60% RH, samples were analyzed for chemical stability by NMR and HPLC. NMR spectra demonstrated that no chemical shift of remdesivir peaks and no new peaks were observed in F10, F12 and F13. Additionally, no degradant peak was observed in the HPLC chromatograms, which agrees with the NMR spectra. Both analyses indicated that remdesivir was chemically stable without degradation after storage.

### ii. Physical Stability of TFF Remdesivir Compositions

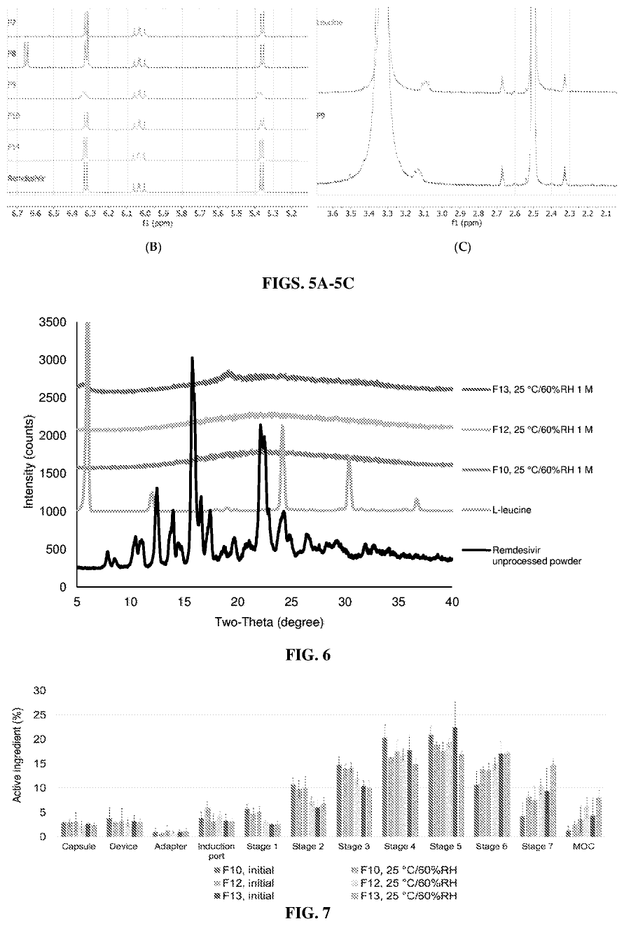

Samples after storage at 25° C./60% RH were analyzed for physical stability by XRD. The XRD diffractogram demonstrates that no remdesivir peaks were observed in any samples after storage (FIG. 6), indicating that remdesivir in F10, F12 and F13 remained amorphous after one month of storage.

### iii. Aerosol Performance after Storage

The aerosol performance of F10, F12 and F13 was analyzed after one month of storage at 25° C./60% RH (Table 3 and FIG. 7). Overall, MMAD and FPF_{<5 μm} (of recovered dose) of F10, F12, and F13 was in the range of 1-2 μm and 74-83%, respectively (Table 3). Although MMAD was slightly decreased in all three formulations, there was no significant change in the aerosol performance after one month of storage at 25° C./60% RH.

TABLE 3
Aerosol performance of TFF remdesivir powders after one month of storage at 25° C./60% RH using
a Plastiape ® RS00 high-resistance DPI at a flow rate of 60 L/min (n = 3). (MMAD, mass
median aerodynamic diameter; GSD, geometric standard deviation; FPF, fine particle fraction; EF, emitted fraction).

| | | | | FPF_{<5 μm} | FPF_{<3 μm} | FPF_{<5 μm} | FPF_{<3 μm} | EF |
|---|---|---|---|---|---|---|---|---|
| | | MMAD | | (%, Recovered | (%, Recovered | (%, Delivered | (%, Delivered | (%, Recovered |
| Formulation | Condition | (μm) | GSD | Dose) | Dose) | Dose) | Dose) | Dose) |
| F10 | Initial | 1.99 ± 0.19 | 2.49 ± 0.13 | 74.84 ± 4.70 | 59.64 ± 4.37 | 80.22 ± 3.08 | 63.93 ± 2.55 | 93.22 ± 2.71 |
| | 25° C./60% | 1.70 ± 0.16 | 2.67 ± 0.13 | 75.85 ± 1.45 | 61.61 ± 2.89 | 80.40 ± 1.79 | 65.31 ± 3.26 | 94.35 ± 0.54 |
| | RH, 1M | | | | | | | |
| F12 | Initial | 1.78 ± 0.23 | 2.72 ± 0.30 | 76.88 ± 6.37 | 62.30 ± 6.15 | 81.79 ± 3.70 | 66.25 ± 4.14 | 93.88 ± 3.61 |
| | 25° C./60% | 1.41 ± 0.20 | 2.65 ± 0.16 | 81.46 ± 2.32 | 69.45 ± 2.83 | 85.89 ± 1.16 | 73.21 ± 2.03 | 94.84 ± 1.52 |
| | RH, 1M | | | | | | | |
| F13 | Initial | 1.33 ± 0.10 | 2.54 ± 0.42 | 83.07 ± 1.35 | 72.76 ± 2.81 | 88.19 ± 2.43 | 77.26 ± 3.87 | 94.22 ± 1.09 |
| | 25° C./60% | 1.19 ± 0.22 | 2.96 ± 0.16 | 81.23 ± 4.16 | 72.46 ± 3.07 | 87.60 ± 1.82 | 76.54 ± 3.74 | 94.69 ± 0.61 |
| | RH, 1M | | | | | | | |

### E. Solubility in Simulated Lung Fluid

The solubility of neat remdesivir in SLF was evaluated. The solubility of amorphous remdesivir was about 20 times higher than that of crystalline remdesivir (0.59±0.01 mg/mL vs. 0.03±0.001 mg/mL). The solubility of remdesivir in F10 and F13 was also determined. The solubility of F10 and F13 was 0.60±0.04 mg/mL and 0.57±0.03 mg/mL, respectively, which was similar to the solubility of amorphous neat remdesivir.

### F. Drug Release in Simulated Lung Fluid

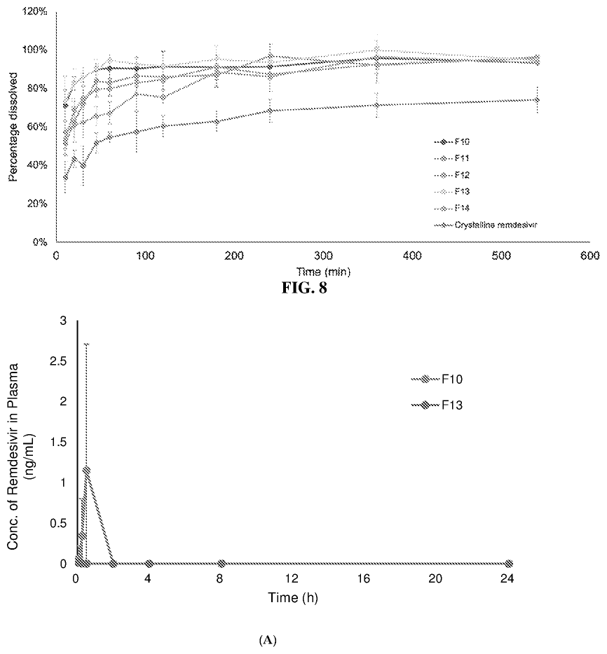

The in vitro dissolution profiles of remdesivir in SLF media under sink conditions are shown in FIG. 8. The dissolution rates of TFF remdesivir formulations were significantly higher than the unprocessed crystalline remdesivir (p<0.05). More than 80% of remdesivir in F10, F11, F12 and F13 was released and dissolved within 90 min, while 80% remdesivir in F14 was dissolved in 180 min.

### G. In Vivo Pharmacokinetic Study for Sprague-Dewey Rats

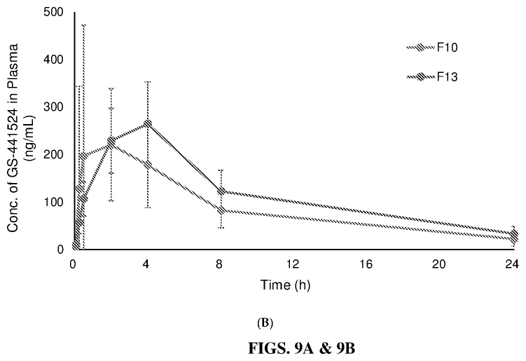

The plasma concentration-time profiles of remdesivir and GS-441524 following a single inhalation dose of TFF remdesivir formulations are shown in FIGS. 9A & 9B, respectively. Remdesivir was detected in plasma at 5 min (0.068 ng/mL) and reached a maximum 30 min (1.149 ng/mL) following pulmonary dosing of F10. A much smaller amount of remdesivir (<0.01 ng/mL) was detected at 5 and 15 min following pulmonary dosing of F13. Remdesivir concentrations were below the lower limit of quantitation in plasma after 2 h and after 30 min following pulmonary dosing of F10 and F13, respectively.

FIG. 9B shows a comparison of plasma GS-441524 concentration-time profile from F10 and F13, and the calculated pharmacokinetic parameters following pulmonary administration are presented in Table 4. The plasma GS-441524 profiles following pulmonary dosing of F10 and F13 has peak concentrations (Cₘₐₓ) of 220.4±118.0 ng/mL at 2 h and 264.3±88.5 ng/mL at 4 h, respectively, before plasma concentrations started decreasing. The AUC₀₋₂₄ and AUC_{inf} of F13 were significantly greater than those of F10 (p<0.05).

Table 5 shows the amount of remdesivir and GS-441524 that remained in the lung tissues of treated animals at 24 h following pulmonary administration of F10 and F13. Despite no statistical difference, F10 exhibited greater amounts of remdesivir in the lungs at 24 h, while F13 showed greater amounts of GS-441524 in the lungs at 24 h.

TABLE 4
In vivo pharmacokinetic parameters
for the plasma GS-441524 concentrations
of F10 and F13 following a
single inhalation administration.

| | Tₘₐₓ | Cₘₐₓ | AUC₀₋₂₄ | AUC_{inf} |
|---|---|---|---|---|
| Formulations | (h) | (ng/mL) | (ng · h/mL) | (ng · h/mL) |
| F10 | 2 | 220.4 ± 118.0 | 2115.3 ± 979.6 | 2397.8 ± 1178.3 |
| F13 | 4 | 264.3 ± 88.5 | 2788.5 ± 857.1 | 3204.9 ± 967.6 |

TABLE 5
Amount of remdesivir and GS-441524 in the lungs at 24 h following
a single inhalation administration of F10 and F13.

| | | | Remdesivir | | GS-441524 | |
|---|---|---|---|---|---|---|
| | | | Total | | Total | |
| | | Wet Lung | Weight | In wet Lung | Weight | In wet Lung |
| Formulation | Rat | (mg) | (μg) | (ng/mg) | (μg) | (ng/mg) |
| F10 | A | 1164.5 | 16.5 | 14.2 | 6.2 | 5.3 |
| | B | 1494.8 | 11.3 | 7.5 | 6.4 | 4.3 |
| | C | 1255.2 | 13.6 | 10.9 | 10.8 | 8.6 |
| | D | 1194.0 | 0.1 | 0.1 | 0.2 | 0.2 |
| | E | 1176.3 | 16.2 | 13.8 | 12.2 | 10.4 |
| | Average ± SD | — | 11.6 ± 6.7 | 9.3 ± 5.8 | 7.2 ± 4.7 | 5.8 ± 4.0 |
| F13 | F | 1025.1 | 12.4 | 11.2 | 12.5 | 11.3 |
| | G | 1126.8 | 7.7 | 7.6 | 15.3 | 14.9 |
| | H | 1254.5 | 4.0 | 3.5 | 18.7 | 16.6 |
| | I | 1263.1 | 3.4 | 2.7 | 18.7 | 14.9 |
| | J | 1164.5 | 0.6 | 0.5 | 1.2 | 1.0 |
| | Average ± SD | — | 5.6 ± 4.5 | 5.1 ± 4.3 | 13.4 ± 7.3 | 11.7 ± 6.3 |

### H. In Vivo Pharmacokinetic Study for Syrian Hamster

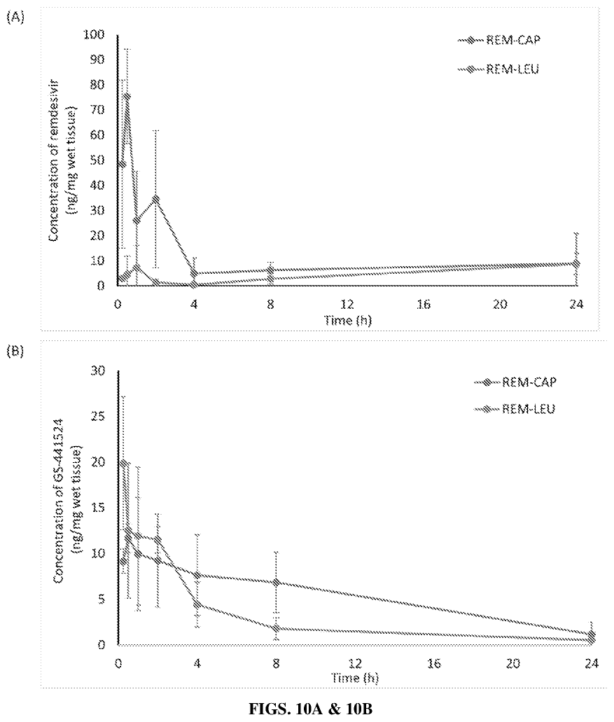

The concentrations of remdesivir and GS-441524 were determined in plasma and lung tissue from healthy hamsters treated with a single 10 mg/kg remdesivir dry powder insufflation of remdesivir/Captisol (REM-CAP) or remdesivir/leucine (REM-LEU) formulation. The resulting lung tissue concentration-versus-time curves are shown in FIG. 10, while the corresponding pharmacokinetic parameters are summarized in Table 6. The Cₘₐₓ of remdesivir for REM-CAP was 8-fold higher than that of REM-LEU (75.41 ng/mg VS 8.71 ng/mg, respectively), while Tₘₐₓ of remdesivir for REM-CAP was lower compared to REM-LEU (30 mins VS 24 hour, respectively). Additionally, REM-CAP exhibited higher AUC₀₋₂₄ of remdesivir in the lungs than REM-LEU, indicating higher absorption of remdesivir in the lung.

TABLE 6
In vivo pharmacokinetic parameters for lung remdesivir and
GS-441524 concentration of REM-CAP and REM-LEU
following a single 10 mg/kg inhalation administration.

| Pharma- | | | | |
|---|---|---|---|---|
| cokinetic | REM-CAP | | REM-LEU | |
| Parameters | Remdesivir | GS-441524 | Remdesivir | GS-441524 |
| T_{1/2} (h) | — | 7.38 | — | 7.12 |
| Tₘₐₓ (h) | 0.5 | 0.5 | 24 | 0.25 |
| Cₘₐₓ (ng/mg) | 75.41 | 11.68 | 8.71 | 19.88 |
| AUC₀₋₂₄ | 260.30 | 128.61 | 109.16 | 71.39 |
| (ng · h/mL) | | | | |
| AUC_{inf} | — | 141.11 | — | 76.85 |
| (ng · h/mL) | | | | |
| MRT_{inf} (h) | — | 9.46 | — | 6.94 |
| V/F (mg/kg) | — | 0.75 | — | 1.34 |
| (ng/mg) | | | | |
| Cl/F ((mg/kg)/ | 0.00147 | 0.00345 | 0.00140 | 0.00209 |
| (ng/mg)/h) | | | | |

For the level of a metabolite in the lungs, the AUC₀₋₂₄ of GS-441524 of REM-CAP was about 1.7 times higher than that of REM-LEU (128.61 ng·h/mg VS 71.39 ng·h/mg, respectively). Despite this, REM-LEU exhibited a higher Cₘₐₓ of GS-441524 (19.88 ng/mg VS 11.68 ng/mg) and shorter Tₘₐₓ of GS-441524 (15 mins VS 30 mins) as compared to REM-CAP. GS-441524, in both formulations, was eliminated in a biphasic pattern with a distribution phase and an elimination phase. The half-life of GS-441524 for both formulations are similar (7.12 hours for REM-CAP and 7.38 hours for REM-LEU).

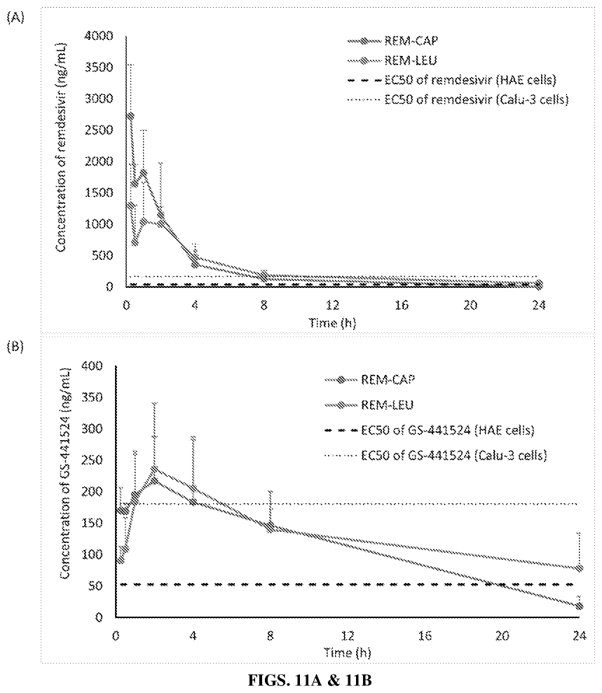

The systemic in vivo pharmacokinetics of drug absorption from the lungs was also investigated in hamsters. FIG. 11 shows the comparison of the mean remdesivir and GS-441524 plasma concentration-time profile from each formulation. The pharmacokinetic parameters following a single dose dry powder insufflation calculated using a non-compartment model are presented in Table 7. Overall, both formulations have similar plasma profiles of remdesivir and GS-441524. Rapid remdesivir concentration decay was observed in both formulations 30 mins following pulmonary administration before reaching the elimination phase. Similarly, GS-441524 plasma concentration for both formulations reached the maximum at 2 hours, and continuously decreased after 2 hours following pulmonary insufflation.

TABLE 7
In vivo pharmacokinetic parameters for plasma remdesivir
and GS-441524 concentration of REM-CAP and REM-LEU
following a single 10 mg/kg inhalation administration.

| Pharma- | | | | |
|---|---|---|---|---|
| cokinetic | REM-CAP | | REM-LEU | |
| Parameters | Remdesivir | GS-441524 | Remdesivir | GS-441524 |
| T_{1/2} (h) | 3.65 | 6.04 | 5.62 | 14.26 |
| Tₘₐₓ (h) | 0.25 | 2 | 0.25 | 2 |
| Cₘₐₓ (ng/mg) | 2726.74 | 217.10 | 1298.65 | 235.78 |
| AUC₀₋₂₄ | 6779.83 | 2736.11 | 6647.90 | 3191.93 |
| (ng · h/mL) | | | | |
| AUC_{inf} | 6818.37 | 2895.69 | 7139.81 | 4792.48 |
| (ng · h/mL) | | | | |
| MRT_{inf} (h) | 3.26 | 8.11 | 7.29 | 21.03 |
| V/F (mg/kg) | 0.0077 | 0.0301 | 0.0113 | 0.0429 |
| (ng/mg) | | | | |
| Cl/F ((mg/kg)/ | 0.0015 | 0.0035 | 0.0014 | 0.0021 |
| (ng/mg)/h) | | | | |

Although similar absorption patterns of remdesivir and GS-441524 were observed, the pharmacokinetic parameters were slightly different. REM-CAP exhibited higher mean remdesivir plasma concentration 15 mins following pulmonary administration when compared to REM-LEU (2726.74 ng/mL VS 1298.65 ng/mL, respectively). However, both formulations have similar AUC₀₋₂₄ and AUC_{0-inf} of remdesivir, indicating a similar extent of drug absorption into the systemic circulation.

For GS-441524 plasma levels, although no significant difference in Cₘₐₓ and Tₘₐₓ of GS-441524 between two formulations was observed, the AUC₀₋₂₄ of GS-441524 from REM-LEU was slightly higher than that of REM-CAP (3192.93 ng/mL VS 2736.11 ng/mL, respectively). Similarly, REM-LEU showed higher AUC_{0-inf} of GS-441524 compared to REM-CAP (4792.48 ng/mL VS 2895.69, respectively).

Interestingly, the half-life of remdesivir from REM-LEU was longer than that of REM-CAP (5.62 VS 3.65 hours, respectively). Likewise, REM-LEU exhibited a longer half-life of GS-441524 in plasma, compared to REM-CAP (14.26 h VS 6.04 h, respectively). This agrees with the mean residence time (MRT) of remdesivir and GS-441524. REM-LEU exhibited longer MRT_{0-inf} of remdesivir and GS-441524, indicating remdesivir and GS-441524 were cleared from the plasma more slowly than from REM-CAP formulation.

The in vivo pharmacokinetic results showed that pulmonary administration can produce plasma concentrations that achieve higher than the 50% maximal effective concentration (EC₅₀). The EC₅₀ is used to quantify the in vitro antiviral efficacy of drugs. Since the activity of antiviral agents depends on the cell type used for viral propagation, viral isolate, and viral quantification (Rasmussen et al., 2020), several EC₅₀ values of remdesivir and GS-441524 against SARS-CoV-2 in different cell lines are reported (Agostini et al., 2018; Pruijssers et al., 2020; Sheahan et al., 2017; Wang et al., 2020). Agostini et al. reported EC₅₀ of remdesivir and GS-441524 against SAR-CoV-2 in human airway epithelial (HAE) to 70 nM (42.18 ng/mL) and 180 nM (52.43 ng/mL), respectively (Agostini et al., 2018). According to the prescribing information of Veklury®, the EC₅₀ of remdesivir in HAE cells and continuous human lung epithelial cell line (Calu-3) is 9.9 nM (5.97 ng/mL) and 280 nM (168.73 ng/mL), respectively (Gilead Sciences, Inc., 2020). Pruijssers et al. also reported that the EC₅₀ of remdesivir and GS-441524 in Calu-3 2B4 cells is 280 nM (168.73 ng/mL), and 620 nM (180.6 ng/mL), respectively (Pruijssers et al., 2020). Despite differences in the reported EC₅₀ values, plasma concentrations following dry powder pulmonary insufflation of both formulations were higher than these reported EC₅₀ values for remdesivir and GS-441524 in HAE cells at least over 20 hours, and higher than the remdesivir EC₅₀ in Calu-3 cells for over 8 hours and likewise 4 hours for GS-441524.

The plasma GS-441524 concentration-time profiles in the hamster study are consistent with the previous pharmacokinetic study in rats (Sahakipijarn et al., 2020a). In the rat PK study, the average Cₘₐₓ of REM-CAP and REM-LEU was in the range of 220-264 ng/L. Likewise, the AUC₀₋₂₄ and AUC_{inf} of both formulations in the rats were in the range of 2115.3-2778.5 ng·h/mL, and 2397.8-3204.9 ng·h/mL, respectively, which are close to the values in our hamster study. Despite the fact that different species have different drug metabolism rates, both studies indicated that pulmonary administration of remdesivir can achieve GS-441524 concentrations higher than the EC₅₀.

The in vivo pharmacokinetic results also provide useful information about dosing interval and dosing regimen. GS-441524 remained in the lungs for about 8 hours before plateauing (FIG. 10B). The half-life of GS-441524 in the lungs of both formulations was about 7 hours.

An effect of excipients on the pharmacokinetics parameters was also observed in this study. REM-CAP exhibited a faster and greater absorption of remdesivir in the lung, as the results showed shorter Tₘₐₓ, greater Cₘₐₓ, and higher AUC of remdesivir in the lung. Additionally, REM-CAP produced a greater Cₘₐₓ of remdesivir in plasma when compared to REM-LEU. As reported in the previous study, the presence of Captisol® resulted in the faster absorption of remdesivir compared to the leucine formulation (Sahakipijarn et al., 2020a). This is possibly related to the properties of Captisol®. As reported in the literature, Captisol® is a sulfobutyl ether derivative of β-cyclodextrin that can produce complexes with poorly soluble lipophilic drugs, and therefore enhance aqueous solubility and dissolution (Lockwood et al., 2003), and the bioavailability of drugs (Beig et al., 2015). Since REM-CAP contains remdesivir and Captisol® in an 80/20 weight ratio (10:1 molar ratio of remdesivir: Captisol®), without wishing to be bound by any theory it is believed that approximately 10% of the remdesivir (on a weight basis) is complexed with Captisol® in a solubilized form (Sahakipijarn et al., 2020a).

Interestingly, the current study demonstrated that the complexation of Captisol® and remdesivir appeared only to have an effect on the absorption rate, but not on the extent of drug absorption into the systemic circulation. The slower lung absorption of remdesivir of REM-LEU did not affect the absorption into systemic circulation as both formulations showed similar AUC_{0-24 h} and Tₘₐₓ of remdesivir in plasma. Moreover, in terms of the efficacy of antiviral agents, pulmonary administration of REM-LEU can produce a higher Cₘₐₓ of GS-441524 with a shorter Tₘₐₓ and a lower AUC of GS-441524, indicating a lower total exposure of GS-441524 is needed to produce high GS-441524 levels in the lung for inhibiting virus replication.

### III. Discussion

### A. Thin Film Freezing Produces High Potency Remdesivir Dry Powders for Inhalation with High Aerosol Performance

The feasibility of using the thin film freezing process to prepare inhalable remdesivir powder formulations was investigated. Different excipients, including Captisol®, mannitol, lactose, and leucine, were evaluated in this study. Mannitol and lactose are presently contained in FDA approved inhalation dosage forms, while leucine has gained recent interest for pulmonary delivery and is being used in an inhaled product in FDA clinical trials (Pilcer et al., 2010; Securities and Exchange Commission, 2019; National Library of Medicine, 2017). Captisol® was selected in this study since it is a solubilizer used in both the solution concentrate for dilution and infusion and the lyophilized powder for reconstitution/dilution and infusion of current remdesivir products (Summary on Compassionate Use: Remdesivir, 2020).

The RS00 high-resistance monodose DPI is a capsule-based DPI device that is available for commercial product development, and it functions to disperse the powder based on impaction force. A previous study confirmed that this impact-based DPI can disperse low-density brittle matrix powders made by TFF process into respirable particles better than a shear-based DPI (e.g., Handihaler®) (Sahakijpijarn et al., 2020). Another study also evaluated the performance of different models of the monodose DPI (RS01 and RS00) on the aerosol performance of brittle matrix powders containing voriconazole nanoaggregates prepared by TFF (Moon et al., 2019). It was shown that the RS00 device exhibited better powder shearing and deaggregation through smaller holes of the capsule created by the piercing system of the RS00 device (Moon et al., 2019). Therefore, the RS00 high-resistance Plastiape® DPI was selected for this study.

The excipient type and drug loading were found to affect the aerosol performance of TFF remdesivir powder compositions. Overall, the aerosol performance of TFF remdesivir powders increased as the drug loading was increased, a highly desirable feature. This trend is obvious for the Captisol®-, lactose-, and mannitol-based formulations when the drug loading was increased from 20% to 50%. Furthermore, high-potency TFF remdesivir powder without excipients (F14 and F15) also exhibited high FPF and small MMAD, which indicates that remdesivir itself has a good dispersing ability without the need of a dispersing excipient when prepared using the TFF process. This shows that the TFF technology can be used to minimize the need of excipient(s) in the formulation, thus maximizing the amount of remdesivir being delivered to the lungs by dry powder inhalation.

The aerosol performance of the leucine-based formulations did not significantly change when the drug loading was increased from 20% to 80%, and these formulations exhibited superior aerosol performance compared to the other excipient-based formulations studied. This is likely attributed to the surface modifying properties of leucine. Leucine can minimize the contact area and distance between particles which can decrease Van der Waal forces between drug particles and subsequently increases aerosol performance (Paajanen et al., 2009; Mangal et al., 2019).

Additionally, different co-solvent systems affected the aerosol performance of TFF remdesivir powders. The formulations, except for the TFF remdesivir-leucine, prepared in a 1,4-dioxane/water co-solvent system exhibited smaller MMAD and higher FPF than the formulations prepared in an acetonitrile/water co-solvent system. Interestingly, the co-solvent systems did not affect the aerosol performance of the TFF remdesivir-leucine formulation. This agrees with SEM figures showing that the formulations prepared in a 1,4-dioxane/water co-solvent system have smaller nanostructured aggregates than the formulations prepared in an acetonitrile/water cosolvent system. This may be due to the difference in the viscosity of the solvent system. The viscosity of acetonitrile/water (50/50 v/v) was lower than that of 1,4-dioxane/water (50/50 v/v) (0.81 vs. 1.62 mPa·s) (Thompson et al., 2006; Besbes et al., 2009). The viscosity of the solvent system has an impact on the aerosol performance of TFF powder (Beinborn et al. (Beinborn et al., 2012) and Moon et al. (Moon et al., 2019)). The higher viscosity of the co-solvent minimizes the movement of molecules during the ultra-rapid freezing step, resulting in more homogenous distribution in the frozen state (Moon et al., 2019). The lower-viscosity solvent allows molecules to move more readily, which increases the chance of molecular aggregation and subsequently decreases the aerosol performance (Moon et al., 2019).

### B. Physical and Chemical Stability of Remdesivir Dry Powder Produced Using TFF

Both XRD diffractograms and DSC thermograms showed that remdesivir was amorphous after the TFF process. An amorphous form of the drug generally provides for faster dissolution rate than its crystalline form. Since the drug needs to be absorbed and then penetrate through the cell membrane before it is hydrolyzed to nucleoside monophosphate GS-441524, TFF remdesivir powders provide benefits for the dissolution of the deposited powder in the lung fluid that leads to improvement in the bioavailability and efficacy of the drug when administered by inhalation.

One potential concern related to the amorphous drug is physical instability due to its high-energy state. According to criteria described by Wyttenbach et al., this study confirmed that remdesivir is categorized as a class III glass-forming drug (it is a stable glass former) (Wyttenbach et al., 2017), because no crystallization peak was observed in both the cooling and heating cycles on DSC. It was reported that the glass-forming ability (GFA) has an influence on the physical stability of drug (Blaabjerg et al., 2019). The GFA class III drugs have a strong tendency to transform into its amorphous state, resulting in the highest physical stability compared to class I and class II active pharmaceutical ingredients (Blaabjerg et al., 2019; Panini et al., 2019).

Additionally, NMR spectra demonstrated that Captisol® and leucine have interactions with remdesivir, which may help to stabilize remdesivir during storage. Moreover, DSC thermograms indicated that lactose and remdesivir were not molecularly dispersed in one single amorphous phase. To investigate the effects of the drug and excipient interactions on physical and chemical stability, F10, F12 and F13 were selected in the stability study.

The properties of a class III glass-forming drug were in agreement with the physical stability of remdesivir. After one month of storage at 25° C./60% RH, the results showed that remdesivir in all three formulations was physically stable. Different types of excipients did not affect the physical stability of remdesivir. Even though lactose was not molecularly dispersed with remdesivir, its use did not affect the physical stability of remdesivir. Lastly, these results indicate that amorphous remdesivir was physically stable without a stabilizer.

In addition to the physical stability, remdesivir, as a prodrug, is prone to degrade by hydrolysis in aqueous solution. Since an organic/water co-solvent system is required to dissolve the drug and excipients in the TFF process, chemical stability is another concern during preparation. NMR spectra demonstrated that remdesivir did not chemically degrade as a result of the TFF process. Even though remdesivir was exposed to binary co-solvent systems consisting of water during the process, the entire TFF process used to produce remdesivir dry powder inhalation formulations did not induce chemical degradation of the parent prodrug. Furthermore, remdesivir was chemically stable by HPLC and NMR after one month of storage at 25° C./60% RH.

### C. Remdesivir in TFF Powder Compositions Can Be Dissolved, Absorbed, and Metabolized to GS-441524 in the Lungs

Since remdesivir is a poorly water-soluble drug, its dissolution may be a critical factor of drug release in the lung fluid, especially in high drug load formulations. Undissolved particles can be cleared by mucociliary clearance or macrophage uptake, causing lower drug concentration, lung irritation, and inflammatory response (Jones and Neef, 2011). Therefore, a dissolution test was evaluated. The dissolution profile demonstrated that physical form of drug appears to have a significant effect on the dissolution rate. Amorphous remdesivir prepared using TFF had a much faster dissolution rate in simulated lung fluid compared to crystalline remdesivir. This agrees with the solubility of remdesivir. Amorphous neat remdesivir showed a 20-fold increase in solubility compared to crystalline neat remdesivir. Moreover, the high porosity and high surface area of the TFF powders also contribute to faster wetting, thereby enhancing the dissolution rate (Hassoun et al., 2018; Sinswat et al., 2008).

The presence of excipients also affected the dissolution rate of remdesivir. The dissolution rate of 100% drug load formulation (no excipient was used), even though it is amorphous, was slower, compared to 80% drug loading formulations. The enhancement of dissolution rate is likely attributed to improved wetting from the use of excipients. Lactose and mannitol are hydrophilic carriers that can wet the powder formulations rapidly, promoting faster exposure of the drug particles to the dissolution medium (Jiang et al., 2020). In the case of Captisol®, cyclodextrins and their derivatives have been widely used as solubility and dissolution modifying excipients. The molar ratio of remdesivir to Captisol® in the high drug load composition (80/20 w/w) was 10:1, meaning that only about 10% of remdesivir was present in a complex with Captisol® in the solubilized form; the remaining remdesivir was not complexed with Captisol® and was present as amorphous powder in the matrix. It is known that certain drugs form a complex with Captisol® by hydrophobic interactions (Stella and Rajewski, 2020; Pal et al., 2020; Beig et al., 2015). The inclusion complexation between cyclodextrin and a drug can generally increase the aqueous solubility of the aforementioned drug, and increase the dissolution rate (van der Merwe et al., 2020). It was previously reported that the solubility of remdesivir can be increased by Captisol®, and Veklury®, and the commercial product containing remdesivir includes Captisol® to solubilize remdesivir for the IV solution formulation (Stella and Rajewski, 2020). However, as used here, 10% complexation of remdesivir and Captisol® in F10 appeared to have an effect on dissolution rate, but it did not affect the solubility of remdesivir since there was no significant difference in the solubility between F10 and amorphous neat remdesivir.

Interestingly, the TFF leucine-based formulation exhibited a higher dissolution rate than the TFF remdesivir formulations that contain mannitol or lactose, despite its hydrophobic properties. The faster dissolution rate of the leucine-based formulation is likely attributed to interactions (e.g., hydrogen bonding) between remdesivir and leucine. Peaks corresponding to acidic protons (—OH or —NH) of remdesivir from F9, a leucine-based formulation, became broader than those from the other formulations without leucine, and shifted downfield. These wider peaks result from inter-molecular interactions (Maity et al., 2019). A peak shift in NMR spectra indicates magnetic field changes. The downfield shift of the acidic protons from F9 is caused by deshielded protons and reduced electron density, indicating hydrogen bonding at the protons (Hibbert and Emsley, 1990; Konrat et al., 1990). Along with a leucine-corresponding peak shift from F9, it was concluded that remdesivir favorably interacts with leucine in hydrogen bonding. The molar ratio of remdesivir to leucine of the high drug load TFF compositions (80/20 w/w) was 1:1.5, meaning that leucine in this composition was in excess in terms of its ability to interact with remdesivir.

Significantly higher plasma levels of GS-441524 than remdesivir were observed in our rat pharmacokinetic study. The half-life of remdesivir is reportedly much shorter than that of GS-441524. While the half-life of GS-441524 is approximately 24.5 h, for remdesivir it is only about 1 h in humans following multiple IV administrations once a day (Summary of Compassionate Use: Remdesvivir Gilead). In rats, the half-life of remdesivir in plasma is reported to be less than 0.9 min, which is much shorter than that in humans, due to higher esterase activity in rodent plasma (Summary of Compassionate Use: Remdesvivir Gilead). Therefore, with a Tₘₐₓ of 30 min and a level of remdesivir in plasma that is lower than the detection limit at 2 h, F10 presented an initial rapid systemic absorption of remdesivir that was complete at less than 2 h. Tₘₐₓ of GS-441524 plasma concentration (2 h) also supports this observation. In comparison, remdesivir plasma concentrations of F13 were lower than those of F10 at 5 and 15 min, and it was lower than the detection limit at the other time points, indicating that the initial systemic absorption of remdesivir from lung was much slower with F13. With the later Tₘₐₓ of GS-441524 plasma concentration, however, more sustained release of remdesivir from lung to plasma was observed.

The initial faster systemic absorption of F10 from the lungs is likely due to the fact that F10 contains about 10% remdesivir (on a molar basis) that is complexed with Captisol® in a solubilized form. Higher concentrations of both remdesivir and GS-441524 were detected in plasma following administration of F10, which contains 20% w/w of Captisol®. Captisol® (sulfobutylether-β-cyclodextrin), is known to enhance the stability, solubility, and bioavailability of drugs (Lockwood et al., 2003). When Captisol® forms a complex by hydrophobic interactions with drugs, these interactions are even greater than with simple physical mixtures of drug and Captisol® (Pal et al., 2020; Beig et al., 2015). Although F10 and F13 consist of amorphous remdesivir in the TFF remdesivir powder formulations, the inclusion of Captisol® in F10 likely increased the absorption of the complexed remdesivir into systemic circulation due to its improvement in solubilization (Stella and Rajweski, 2020; Yang, 2020). Even though F10 incorporates a high molar ratio of drug to Captisol® (10:1), and not all of the remdesivir in F10 complexes with Captisol®, a partial remdesivir-Captisol® complex will still dissolve faster in lung fluid, rapidly becoming a solution and being absorbed into systemic circulation more quickly than the non-complexed remdesivir fraction with the Captisol® of F10. Eriksson et al. described that drugs in solution can be absorbed faster than dry powder in lungs in the isolated perfused lung model (Eriksson et al., 2019).

Lastly, and related to potential use in COVID-19 therapy, the 50% maximal effective concentration (EC₅₀) value is important to evaluate the antiviral efficacy of drugs. While there are studies reported to evaluate remdesivir potency to inhibit SARS-CoV-2 replication, only one study has reported antiviral efficacy of GS-441524 against SARS-CoV-2. Pruijssers et al. determined the inhibition of SARS-CoV-2 replication in established cell lines by GS-441524, and reported that EC₅₀ is 0.62 μM (180.6 ng/mL) in Calu3 2B4 cells, and 0.47 μM (136.9 ng/mL) in Vero E6 cells (Pruijssers et al., 2020). The target dose of 10 mg/kg by dry powder insufflation in the rat pharmacokinetic study achieved the EC₅₀ values necessary to reach EC₅₀ in order to inhibit SARS-CoV-2. The plasma concentrations of GS-441524 in this pharmacokinetic study also achieved higher than the reported EC₅₀ of 0.18 μM (52.4 ng/mL) in SARS-CoV-infected human airway epithelial cells (Yan and Muller, 2020).

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.

U.S. Pat. No. 8,968,786
U.S. Patent App. No. 2010/0221343
Agostini et al., mBio, 9(2):e00221-18, 2018.

### Ahmed et al., Viruses, 12:254, 2020.

### Annual Report Pursuant to Section 13 or 15(D) of the Securities Act of 1934, Securities and Exchange Commission, 2020.

Arora et al., Pharm. Res., 27:786-795, 2010.
Beig et al., Eur. J. Pharm. Sci., 77:73-78, 2015.
Beigel et al., N. Eng. J. Med., 2020.
Beinborn et al., European journal of pharmaceutics and biopharmaceutics, 81(3):600-8, 2012a.

### Beinborn et al., Int J Pharmaceut., 429(1-2):46-57, 2012b.

Besbes et al., J. Mol. Liq., 145:1-4, 2009.
Blaabjerg et al., J. Pharm. Sci., 108:2561-2569, 2019.
Brunaugh et al., bioRxiv, 310490, 2020.
Cares-Pacheco et al., Int. J. Pharm., 8:1769-1780, 2014.
Carvalho et al., European journal of pharmaceutics and biopharmaceutics, 88(1):136-47, 2014.

### DiNunzio et al., Mol Pharm., 5(6):968-80, 2008.

### Engstrom et al., Pharm Res.; 25(6):1334-46, 2008.

Eriksson et al., Mol. Pharm., 16:3053-3064, 2019.
Gurwitz, D. Drug Dev. Res., 2020.

### Handbook of Pharmaceutical Salts: Properties, and Use (P. H. Stahl & C. G. Wermuth eds., Verlag Helvetica Chimica Acta, 2002).

Hassoun et al., J. Drug. Deliv. Sci. Technol., 47:485-491, 2018.
Hibbert et al., Adv. Phys. Org. Chem., 26:255-379, 1990.

### Jiang et al., Dry Technol., 38:889-902, 2020.

### Jones and Neef, Xenobiotica, 42:86-93, 2011.

Konrat et al., Mon. Fur. Chem., 130:961-982, 1999.

### Lang et al., Mol Pharm., 11(1):186-96, 2014b.

### Lang et al., J Drug Deliv Sci Tec., 24(2):205-11, 2014a.

### Laube et al., European Respiratory J., 37:1308-1417, 2011.

Liu et al., European journal of pharmaceutics and biopharmaceutics, 96:132-42, 2015.
Lockwood et al., J. Pharm. Sci., 92:922-926, 2003.
Longest et al., Pharm. Res., 34:2049-2065, 2017.
Mangal et al., Int. J. Pharm., 568:118504, 2019.
Maity et al., Nat. Prod. Commun., 14:1-17, 2019.
Moon et al., J. Drug Deliv. Sci. Technol., 54:1-11, 2019.

### National Library of Medicine, A Study of AeroVanc for the Treatment of MRSA Infection in CF Patients, 2017.

### O'Donnell et al., Drug Dev Ind Pharm., 39(2):205-17, 2013.

### Overhoff et al., Pharm Res., 25(1):167-75, 2008.

Overhoff et al., European journal of pharmaceutics and biopharmaceutics, 65(1):57-67, 2007b.

### Overhoff et al., Int J Pharmaceut., 336(1):122-32, 2007a.

### Overhoff et al., J Drug Del Sci Tech., 19(2):89-98, 2009.

### Paajanen et al., Powder Technol., 192:6-11, 2009.

### Pal et al., ACS Omega, 5:19968-19977, 2020.

### Panini et al., Pharmaceutics, 11:529, 2019.

Peeri et al., Int. J. Epidemiol., 2020.
Pilcer et al., Int. J. Pharm., 392:1-19, 2010.

### Pruijssers et al., Cell Rep., 32:107940, 2020.

### Purvis et al., Aaps Pharmscitech., 8(3):E58, 2007.

### Rasmussen et al., The AAPS Journal, 22(6):1-2, 2020.

Rohrschneider et al., Mol. Pharm., 12:2618-2624, 2015.
Sahakijpijarn et al., Int. J. Pharm., 586:119490, 2020.
Sheahan et al., Science Translational Medicine, 9(396):eaal3653, 2017.
Sinswat et al., European journal of pharmaceutics and biopharmaceutics, 69(3):1057-66, 2008.
Stella & Rajewski, Int. J. Pharm., 583:119396, 2020.

### Summary on Compassionate Use: Remdesivir Gilead, Amsterdam, The Netherlands, 2020.

### Thakkar et al., Hum Vaccin Immunother, 13(4):936-46, 2017.

Thompson et al., J. Chromatogr. A, 1134:201-209, 2006.
Tortoric et al., Nat. Struct. Mol. Biol., 26:481-489, 2019.

### van der Merwe et al., Pharmaceutics, 12:393, 2020.

### Walls et al., Cell, 2020.

### Wang et al., Aaps Pharmscitech., 15(4):981-93, 2014.

### Wang et al., Cell Res., 30(3):269-271, 2020.

### Watts et al., Int J Pharmaceut., 384(1-2):46-52, 2010.

Watts, et al., Pharm. Res., 30:813-825, 2013.
WHO Director-General's opening remarks at the Mission briefing on COVID-19—12 Mar. 2020. www.who.int/dg/speeches/detail/who-director-general-s-opening-remarks-at-the-mission-briefing-on-covid-19-12-march-2020.
Wu et al., J. Antimicrob. Agents Chemother., 48, 2693-2696, 2004.
Wyttenbach et al., Eur. J. Pharm. Biopharm., 112:204-208, 2017.

### Yan et al., Science, 2020.

Yan et al., ACS Med. Chem. Lett., 11:1361-1366, 2020.

### Yang et al., 2010.

Yang et al., Dlin. Transl. Sci., 13:842-844, 2020.
Zhang et al., European journal of pharmaceutics and biopharmaceutics, 82(3):534-44, 2012.

What is claimed is:

1. A pharmaceutical composition comprising:
(A) an active pharmaceutical ingredient wherein active pharmaceutical ingredient is remdesivir or a pharmaceutically acceptable salt thereof;
wherein the pharmaceutical composition is formulated for administration via inhalation and the pharmaceutical composition is a dry powder.

2. The pharmaceutical composition of claim 1, wherein the pharmaceutical composition is a brittle matrix particle.

3. The pharmaceutical composition of either claim 1 or claim 2, wherein the pharmaceutical composition comprises one or more nanoparticles.

4. The pharmaceutical composition according to any one of claims 1-3, wherein the pharmaceutical composition comprises at least 75% of the active pharmaceutical ingredient in an amorphous form.

5. The pharmaceutical composition according to any one of claims 1-4, wherein at least 90% of the active pharmaceutical ingredient is in the amorphous form.

6. The pharmaceutical composition according to any one of claims 1-5, wherein at least 95% of the active pharmaceutical ingredient is in the amorphous form.

7. The pharmaceutical composition according to any one of claims 1-6, wherein at least 98% of the active pharmaceutical ingredient is in the amorphous form.

8. The pharmaceutical composition according to any one of claims 1-7, wherein at least 99% of the active pharmaceutical ingredient is in the amorphous form.

9. The pharmaceutical composition according to any one of claims 1-8, wherein the pharmaceutical composition comprises no crystalline active pharmaceutical ingredient.

10. The pharmaceutical composition according to any one of claims 1-9, wherein the pharmaceutical composition further comprises an excipient.

11. The pharmaceutical composition of claim 10, wherein the excipient is in the crystalline form.

12. The pharmaceutical composition of claim 10, wherein the excipient is in the amorphous form.

13. The pharmaceutical composition according to any one of claims 10-12, wherein the excipient is an amino acid.

14. The pharmaceutical composition of claim 13, wherein the amino acid is a hydrophobic amino acid.

15. The pharmaceutical composition of claim 14, wherein the amino acid is leucine.

16. The pharmaceutical composition according to any one of claims 10-12, wherein the excipient is a sugar or sugar derivative.

17. The pharmaceutical composition of claim 16, wherein the sugar is a sugar alcohol.

18. The pharmaceutical composition of claim 17, wherein the sugar alcohol is mannitol.

19. The pharmaceutical composition of claim 16, wherein the sugar is a monosaccharide or disaccharide.

20. The pharmaceutical composition of claim 19, wherein the sugar is a disaccharide.

21. The pharmaceutical composition of claim 20, wherein the sugar is lactose.

22. The pharmaceutical composition according to any one of claims 10-12, wherein the excipient is a cyclodextrin.

23. The pharmaceutical composition of claim 22, wherein the cyclodextrin is a β-cyclodextrin.

24. The pharmaceutical composition of either claim 22 or claim 23, wherein the cyclodextrin is modified with one or more sulfonate groups.

25. The pharmaceutical composition of claim 24, wherein the sulfonate groups are sulfonate salts.

26. The pharmaceutical composition of claim 25, wherein the sulfonate salts are alkali metal salts.

27. The pharmaceutical composition of claim 26, wherein the sulfonate salts are sodium salts.

28. The pharmaceutical composition according to any one of claims 24-27, wherein the cyclodextrin and the sulfonate groups are connected by an ether spacer.

29. The pharmaceutical composition of claim 28, wherein the ether spacer is a butyl ether spacer.

30. The pharmaceutical composition according to any one of claims 22-29, wherein the excipient is Captisol® or Dexolve™.

31. The pharmaceutical composition according to any one of claims 1-30, wherein the pharmaceutical composition comprises from about 1% w/w to about 99% w/w of the active pharmaceutical ingredient.

32. The pharmaceutical composition according to any one of claims 1-31, wherein the pharmaceutical composition comprises from about 5% w/w to about 95% w/w of the active pharmaceutical ingredient.

33. The pharmaceutical composition according to any one of claims 1-32, wherein the pharmaceutical composition comprises from about 10% w/w to about 90% w/w of the active pharmaceutical ingredient.

34. The pharmaceutical composition of claim 33, wherein the pharmaceutical composition comprises from about 5% w/w to about 45% w/w of the active pharmaceutical ingredient.

35. The pharmaceutical composition of claim 33, wherein the pharmaceutical composition comprises from about 45% w/w to about 90% w/w of the active pharmaceutical ingredient.

36. The pharmaceutical composition according to any one of claims 1-35, wherein the pharmaceutical composition comprises from about 5% w/w to about 95% w/w of the excipient.

37. The pharmaceutical composition of claim 36, wherein the pharmaceutical composition comprises from about 5% w/w to about 45% w/w of the excipient.

38. The pharmaceutical composition of claim 36, wherein the pharmaceutical composition comprises from about 45% w/w to about 90% w/w of the excipient.

39. The pharmaceutical composition according to any one of claims 1-38, wherein the pharmaceutical composition is substantially free of any other compound other than the active pharmaceutical ingredient.

40. The pharmaceutical composition of claim 39, wherein the pharmaceutical composition is essentially free of any other compound other than the active pharmaceutical ingredient.

41. The pharmaceutical composition of claim 40, wherein the pharmaceutical composition is entirely free of any other compound other than the active pharmaceutical ingredient.

42. The pharmaceutical composition according to any one of claims 1-41, wherein the pharmaceutical composition has a median mass aerodynamic diameter from about 0.1 μm to about 10 μm.

43. The pharmaceutical composition of claim 42, wherein the median mass aerodynamic diameter is from about 0.25 μm to about 5 μm.

44. The pharmaceutical composition of claim 43, wherein the median mass aerodynamic diameter is from about 0.5 μm to about 3.5 μm.

45. The pharmaceutical composition of claim 44, wherein the median mass aerodynamic diameter is from about 0.75 μm to about 3 μm.

46. The pharmaceutical composition according to any one of claims 1-45, wherein the pharmaceutical composition comprises a geometric standard deviation is from about 0.1 to about 5.

47. The pharmaceutical composition of claim 46, wherein the geometric standard deviation is from about 0.5 to about 4.5.

48. The pharmaceutical composition of claim 47, wherein the geometric standard deviation is from about 1 to about 4.

49. The pharmaceutical composition of claim 48, wherein the geometric standard deviation is from about 2 to about 3.75.

50. The pharmaceutical composition of claim 49, wherein the geometric standard deviation is from about 2 to about 3.

51. The pharmaceutical composition according to any one of claims 1-50, wherein the pharmaceutical composition has a delivered fine particle fraction of greater than 50% when formulated into an inhaler.

52. The pharmaceutical composition of claim 51, wherein the delivered fine particle fraction is greater than 60%.

53. The pharmaceutical composition of claim 52, wherein the delivered fine particle fraction is greater than 70%.

54. The pharmaceutical composition of claim 53, wherein the delivered fine particle fraction is greater than 80%.

55. The pharmaceutical composition according to any one of claims 1-54, wherein the pharmaceutical composition has a recovered fine particle fraction of greater than 50% when formulated into an inhaler.

56. The pharmaceutical composition of claim 55, wherein the recovered fine particle fraction is greater than 60%.

57. The pharmaceutical composition of claim 56, wherein the recovered fine particle fraction is greater than 70%.

58. The pharmaceutical composition of claim 57, wherein the recovered fine particle fraction is greater than 80%.

59. The pharmaceutical composition according to any one of claims 1-54, wherein the pharmaceutical composition has an emitted dose of greater than 75% when formulated into an inhaler.

60. The pharmaceutical composition of claim 59, wherein the emitted dose is greater than 80%.

61. The pharmaceutical composition of claim 60, wherein the emitted dose is greater than 85%.

62. The pharmaceutical composition of claim 61, wherein the emitted dose is greater than 90%.

63. The pharmaceutical composition according to any one of claims 1-62, wherein the pharmaceutical composition is substantially free of any lubricants, antistatic agents, anti-adherents, glidants, peptides, surfactants, lipids, and phospholipids.

64. The pharmaceutical composition of claim 63, wherein the pharmaceutical composition is essentially free of any lubricants, antistatic agents, anti-adherents, glidants, peptides, surfactants, lipids, and phospholipids.

65. The pharmaceutical composition of claim 64, wherein the pharmaceutical composition is entirely free of any lubricants, antistatic agents, anti-adherents, glidants, peptides, surfactants, lipids, and phospholipids.

66. The pharmaceutical composition according to any one of claims 1-9, 31-35, and 39-65, wherein the pharmaceutically composition is substantially free of any added excipients.

67. The pharmaceutical composition of claim 66, wherein the pharmaceutical composition is essentially free of any added excipients.

68. The pharmaceutical composition of claim 67, wherein the pharmaceutical composition is entirely free of any added excipients.

69. The pharmaceutical composition according to any one of claims 1-9, 31-35, and 39-68, wherein the pharmaceutical composition is substantially free of any excipients.

70. The pharmaceutical composition of claim 69, wherein the pharmaceutical composition is essentially free of any excipients.

71. The pharmaceutical composition of claim 70, wherein the pharmaceutical composition is entirely free of any excipients.

72. The pharmaceutical composition according to any one of claims 1-71, wherein the pharmaceutical composition is loaded into an inhaler.

73. The pharmaceutical composition of claim 72, wherein the inhaler is a dry powder inhaler, a metered dose inhaler, a single dose inhaler, a multi-dose inhaler, or a pressurized metered dose inhaler.

74. The pharmaceutical composition according to any one of claims 1-73, wherein the pharmaceutical composition is formulated as unit dose.

75. The pharmaceutical composition of claim 74, wherein the unit dose is formulated for dry powder inhalation as a capsule, cartridge, or blister.

76. The pharmaceutical composition of claim 75, wherein the capsule, cartridge, or blister is designed for use with a dry powder inhaler.

77. The pharmaceutical composition according to any one of claims 1-76, wherein the pharmaceutical composition is present in a container which blocks UV light.

78. The pharmaceutical composition according to any one of claims 1-77, wherein the pharmaceutical composition is present in a container which blocks moisture uptake.

79. The pharmaceutical composition according to any one of claims 1-78, wherein the pharmaceutical composition is present in a container which blocks oxygen ingress.

80. The pharmaceutical composition according to any one of claims 1-79, wherein the pharmaceutical composition is present in a container which desiccates the composition.

81. The pharmaceutical composition according to any one of claims 1-80, wherein the composition further comprises a second active pharmaceutical ingredient.

82. The pharmaceutical composition according to any one of claims 1-81, wherein the second active pharmaceutical ingredient is anti-inflammatory.

83. The pharmaceutical composition of claim 82, wherein the second active pharmaceutical ingredient is beclomethasone, budesonide, dexamethasone, ciclesonide, fluticasone, mometasone, prednisone, methylprednisone, a statin, or clofazimine.

84. The pharmaceutical composition of claim 81, wherein the second active pharmaceutical ingredient is anti-microbial.

85. The pharmaceutical composition of claim 84, wherein the second active pharmaceutical ingredient is niclosamide, ivermectin, chloroquine, hydroxychloroquine, lopinavir, or favipiravir.

86. The pharmaceutical composition of claim 81, wherein the second active pharmaceutical ingredient is an antibody.

87. The pharmaceutical composition of claim 81, wherein the second active pharmaceutical ingredient is an immunomodulatory therapy.

88. The pharmaceutical composition of claim 87, wherein the immunomodulatory therapy is tocilizumab, sarilumab, anakinra, or ruxolitinib.

89. The pharmaceutical composition of claim 81, wherein the second active pharmaceutical ingredient is an anticoagulant.

90. The pharmaceutical composition of claim 89, wherein the anticoagulant is heparin.

91. The pharmaceutical composition of claim 81, wherein the second active pharmaceutical ingredient is an antifibrotic.

92. The pharmaceutical composition of claim 91, wherein the antifibrotic is a tyrosine kinase inhibitor.

93. An inhaler comprising a pharmaceutical composition according to any one of claims 1-92.

94. A method of preparing a dry powder pharmaceutical composition according to any one of claims 1-92 comprising:
(A) dissolving an active pharmaceutical ingredient, wherein the active agent is remdesivir or a pharmaceutically acceptable salt thereof, in a solvent to obtain a pharmaceutical mixture;
(B) applying the pharmaceutical mixture to a surface at a surface temperature below 0° C. to obtain a frozen pharmaceutical mixture; and
(C) collecting the frozen pharmaceutical mixture and drying the frozen pharmaceutical mixture to obtain a dry powder pharmaceutical composition.

95. The method of claim 94, wherein the solvent is an organic solvent.

96. The method of claim 95, wherein the solvent is acetonitrile, tert-butanol, or 1,4-dioxane.

97. The method according to any one of claims 94-96 further comprising admixing the active pharmaceutical ingredient with an excipient.

98. The method according to any one of claims 94-97, wherein the pharmaceutical mixture further comprises a second solvent.

99. The method of claim 98, wherein the excipient is dissolved in the second solvent and then added to the pharmaceutical mixture.

100. The method of either claim 98 or claim 99, wherein the second solvent is water.

101. The method according to any one of claims 94-100, wherein the first solvent is mixed with the second solvent to obtain a homogenous pharmaceutical mixture.

102. The method of either claim 97 or claim 100, wherein the pharmaceutical mixture is admixed until the pharmaceutical mixture is clear.

103. The method according to any one of claims 94-102, wherein the pharmaceutical mixture comprises a solid content from about 0.05% w/v to about 5% w/v of the active pharmaceutical ingredient and the excipient.

104. The method of claim 103, wherein the solid content is from about 0.1% w/v to about 2.5% w/v of the active pharmaceutical ingredient and the excipient.

105. The method of claim 104, wherein the solid content is from about 0.15% w/v to about 1.5% w/v of the active pharmaceutical ingredient and the excipient.

106. The method of claim 105, wherein the solid content is from about 0.2% w/v to about 0.6% w/v of the active pharmaceutical ingredient and the excipient.

107. The method of claim 105, wherein the solid content is from about 0.5% w/v to about 1.25% w/v of the active pharmaceutical ingredient and the excipient.

108. The method according to any one of claims 94-107, wherein the pharmaceutical mixture is applied at a feed rate from about 0.5 mL/min to about 5 mL/min.

109. The method of claim 108, wherein the feed rate is from about 1 mL/min to about 3 mL/min.

110. The method of claim 109, wherein the feed rate is about 2 mL/min.

111. The method according to any one of claims 94-110, wherein the pharmaceutical mixture is applied with a nozzle.

112. The method of claim 111, wherein the nozzle is a needle.

113. The method according to any one of claims 94-112, wherein the pharmaceutical mixture is applied from a height from about 2 cm to about 50 cm.

114. The method of claim 113, wherein the height is from about 5 cm to about 20 cm.

115. The method of claim 114, wherein the height is about 10 cm.

116. The method according to any one of claims 94-115, wherein the surface temperature is from about 0° C. to −190° C.

117. The method of claim 116, wherein the surface temperature is from about −25° C. to about −125° C.

118. The method of claim 117, wherein the surface temperature is about −100° C.

119. The method according to any one of claims 94-118, wherein the surface is a rotating surface.

120. The method of claim 119, wherein the surface is rotating at a speed from about 5 rpm to about 500 rpm.

121. The method of claim 120, wherein the surface is rotating at a speed from about 100 rpm to about 400 rpm.

122. The method of claim 121, wherein the surface is rotating at a speed of about 200 rpm.

123. The method according to any one of claims 94-122, wherein the frozen pharmaceutical composition is dried by lyophilization.

124. The method of claim 123, wherein the frozen pharmaceutical composition is dried at a first reduced pressure.

125. The method of claim 124, wherein the first reduced pressure is from about 10 mTorr to 500 mTorr.

126. The method of claim 125, wherein the first reduced pressure is from about 50 mTorr to about 250 mTorr.

127. The method of claim 126, wherein the first reduced pressure is about 100 mTorr.

128. The method of according to any one of claims 123-127, wherein the frozen pharmaceutical composition is dried at a first reduced temperature.

129. The method of claim 128, wherein the first reduced temperature is from about 0° C. to −100° C.

130. The method of claim 129, wherein the first reduced temperature is from about −20° C. to about −60° C.

131. The method of claim 130, wherein the first reduced temperature is about −40° C.

132. The method according to any one of claims 123-131, wherein the frozen pharmaceutical composition is dried for a primary drying time period from about 3 hours to about 36 hours.

133. The method of claim 132, wherein the primary drying time period is from about 6 hours to about 24 hours.

134. The method of claim 133, wherein the primary drying time period is about 20 hours.

135. The method according to any one of claims 94-134, wherein the frozen pharmaceutical composition is dried a secondary drying time period.

136. The method of claim 135, wherein the frozen pharmaceutical composition is dried a secondary drying time at a second reduced pressure.

137. The method of claim 136, wherein the secondary drying time is at a reduced pressure is from about 10 mTorr to 500 mTorr.

138. The method of claim 137, wherein the secondary drying time is at a reduced pressure is from about 50 mTorr to about 250 mTorr.

139. The method of claim 138, wherein the secondary drying time is at a reduced pressure is about 100 mTorr.

140. The method of according to any one of claims 135-139, wherein the frozen pharmaceutical composition is dried a secondary drying time at a second reduced temperature.

141. The method of claim 140, wherein the second reduced temperature is from about 0° C. to 30° C.

142. The method of claim 141, wherein the second reduced temperature is from about 10° C. to about 30° C.

143. The method of claim 142, wherein the second reduced temperature is about 25° C.

144. The method according to any one of claims 135-143, wherein the frozen pharmaceutical composition is dried for a second time for a second time period from about 3 hours to about 36 hours.

145. The method of claim 144, wherein the second time period is from about 6 hours to about 24 hours.

146. The method of claim 145, wherein the second time period is about 20 hours.

147. A pharmaceutical composition prepared by the method according to any one of claims 94-146.

148. A method of treating a disease or disorder in a patient comprising administering a pharmaceutical composition according to any one of claims 1-92 and 147 to the patient in a therapeutically effective amount.

149. The method of claim 148, wherein the disease or disorder is a microbial infection.

150. The method of claim 149, wherein the microbial infection is a viral infection.

151. The method of claim 150, wherein the viral infection is an infection of a coronavirus.

152. The method of claim 151, wherein the coronavirus is MERS-Cov, SARS-Cov1, or SARS-Cov2 (COVID-19).

153. The method according to any one of claims 148-152, wherein the active pharmaceutical ingredient is inhaled into the lungs.

154. The method of claim 153, wherein the active pharmaceutical ingredient is inhaled into the alveolar sacs within the lungs.

155. The method according to any one of claims 148-154, wherein the patient is exhibiting one or more symptoms of a viral infection.

156. The method according to any one of claims 148-155, wherein the patient is not yet hospitalized.

157. The method according to any one of claims 148-155, wherein the patient has been hospitalized.

158. The method according to any one of claim 148-157, wherein the patient is not yet receiving supplemental oxygen.

159. The method according to any one of claims 148-158, wherein the patient is not yet receiving mechanical ventilation.

160. The method according to any one of claims 148-159, wherein the patient has not yet been diagnosed with a viral infection.

161. The method according to any one of claims 148-159, wherein the patient has been exposed to a person exhibiting one or more symptoms of a viral infection.

162. The method according to any one of claims 148-161, wherein the patient has been exposed to a person who has been diagnosed with a viral infection.

163. The method of claim 162, wherein the patient is administered the pharmaceutical composition prophylactically.

164. The method according to anyone of claims 148-162, wherein the patient has been diagnosed with a viral infection.

165. The method according to anyone of claims 155-164, wherein the viral infection is an infection of a coronavirus.

166. The method of claim 165, wherein the coronavirus is SARS-Cov2.

167. The method according to any one of claims 148-164, wherein the method comprises administering a dose from about 1 mg to about 250 mg.

168. The method of claim 167, wherein the dose is from about 5 mg to about 100 mg.

169. The method of claim 168, wherein the dose is from about 7.5 mg to about 75 mg.

170. The method of claim 169, wherein the dose is from about 10 mg to about 30 mg.

171. A method of reducing lung inflammation in a patient comprising administering a pharmaceutical composition according to any one of claims 1-92 and 147 to the patient in a therapeutically effective amount.

172. The method of claim 171, wherein the lung inflammation is associated with a viral infection.

173. The method according to any one of claims 148-172, wherein the pharmaceutical composition is administered once.

174. The method according to any one of claims 148-172, wherein the pharmaceutical composition is administered more than once.

175. The method according to any one of claim 148-172 or 174, wherein the pharmaceutical composition is administered at a first dose and then administered again at a different second dose.

176. The method of claim 175, wherein the first dose is greater than the second dose.
